(19) United States
(12) Patent Application Publication
(10) Pub. No.: US 2020/0324225 A1
(43) Pub. Date: Oct. 15, 2020

(54) ACOUSTIC TRANSDUCER CONTROLLER CONFIGURATION

(71) Applicant: FloDesign Sonics, Inc., Wilbraham, MA (US)

(72) Inventors: Bart Lipkens, Bloomfield, CT (US); Ronald E. Musiak, Westfield, MA (US); Dane Mealey, Somers, CT (US); John Artis, Santa Rosa, CA (US)

(21) Appl. No.: 16/754,065

(22) PCT Filed: May 4, 2018
(86) PCT No.: PCT/US2018/031267

§ 371 Date: Apr. 6, 2020

Related U.S. Application Data
(63) Continuation of application No. 15/960,451, filed on Apr. 23, 2018, now abandoned.
Application No. 15/960,451 is a continuation of application No. 15/872,984, filed on Jan. 16, 2018.
Application No. 15/872,984 is a continuation of application No. 15/621,691, filed on Jun. 13, 2017, now Pat. No. 10,350,514.
(60) Provisional application No. 62/501,722, filed on May 4, 2017.

(51) Int. Cl. B01D 17/12; B01D 17/06; B01D 21/28; B01D 29/72; C02F 1/36; H04R 1/06; H01L 41/09; H03H 3/02; H03H 9/00; B01D 21/00; B06B 1/06; C12M 1/26; C12M 1/00; A61M 1/36

(52) U.S. Cl. CPC B01D 17/12; B06B 1/0622; B01D 21/28; B01D 29/72; C02F 1/36; H04R 1/06; H01L 41/09; H03H 3/02; H03H 9/009; B01D 21/0045; B01D 21/283; B06B 1/06; C12M 33/08; C12M 47/02; A61M 1/3678; B01D 17/06

(57) ABSTRACT

An RF driver provides power to an acoustic transducer, which can be implemented as a piezoelectric element, which presents a reactive load. The driver can be a linear amplifier or a combination of a DC-DC converter and DC-AC inverter. A controller implements a control technique for efficient transducer operation. The control technique can locate a frequency for operation that is at a reactance minimum or maximum for the transducer to provide efficient operation of that transducer. An implementation of the controller can be provided in modular hardware.

### BACKGROUND

Acoustophoresis is the separation of particles and secondary fluids from a primary or host fluid using acoustics, such as acoustic standing waves. Acoustic standing waves can exert forces on particles in a fluid when there is a differential in density and/or compressibility, otherwise known as the acoustic contrast factor. The pressure profile in a standing wave contains areas of local minimum pressure amplitudes at standing wave nodes and local maxima at standing wave anti-nodes. Depending on their density and compressibility, the particles can be trapped at the nodes or anti-nodes of the standing wave. Generally, the higher the frequency of the standing wave, the smaller the particles that can be trapped.

At a micro scale, for example with structure dimensions on the order of micrometers, conventional acoustophoresis systems tend to use half or quarter wavelength acoustic chambers, which at frequencies of a few megahertz are typically less than a millimeter in thickness, and operate at very slow flow rates (e.g., μL/min). Such systems are not scalable since they benefit from extremely low Reynolds number, laminar flow operation, and minimal fluid dynamic optimization.

At the macro-scale, planar acoustic standing waves have been used in separation processes. However, a single planar wave tends to trap the particles or secondary fluid such that separation from the primary fluid is achieved by turning off or removing the planar standing wave. The removal of the planar standing wave may hinder continuous operation. Also, the amount of power that is used to generate the acoustic planar standing wave tends to heat the primary fluid through waste energy, which may be disadvantageous for the material being processed.

Conventional acoustophoresis devices have thus had limited efficacy due to several factors including heat generation, use of planar standing waves, limits on fluid flow, and the inability to capture different types of materials.

### SUMMARY

Discussed herein are systems and methods for acoustophoresis for generating optimized particle/fluid clusters to improve gravity/buoyancy separation and collection efficiency. Improved, continuous, acoustophoresis devices using improved fluid dynamics are also discussed, as well as drivers and control devices for operating/implementing the systems and methods.

Control of the acoustic transducer can be implemented on the basis of power setpoints. For example, a user can set a desired power level for power delivered to the transducer. Performance of acoustophoresis in an acoustic chamber using the acoustic transducer can be modulated on the basis of modulated input power to the acoustic transducer. In some instances, a power setpoint is desired for operation, while other parameters, such as frequency, for example, are modified. The power setpoint determines the power output of an RF power supply or power amplifier. A power control is provided to maintain the power setpoint, while other parameters associated with operation of the acoustophoresis device are varied. The power control senses signals provided to the acoustic transducer, such as, for example, voltage and current. These feedback signals are used to determine frequency and phase angle for the power delivered to the transducer. In some examples, a buck converter is used as the power supply. The buck converter has a response bandwidth, which may influence the responsiveness of the power control. For example, if the buck converter bandwidth is relatively narrow, the system response for the power control may be relatively slow for the desired operational performance environment for the acoustophoresis device.

A number of different materials may be processed through the acoustophoresis device, each of which may provide different load characteristics on the acoustic transducer and acoustic chamber. The power supply thus may be subjected to a wide range of loads, which may place demands on the power supply that are challenging to meet. For example, heavy loading of the acoustic transducer and/or acoustic chamber experienced with certain types of materials being processed may cause power supply components to be overloaded, and/or overheated, or may cause trip point thresholds to be met or exceeded. The heavy loading or trip point thresholds crossings may cause faults to be identified in the power control, causing the power supply to be shut down. In addition, the power demands on the power supply may change significantly with changes in other operational parameters, such as temperature, frequency or loading characteristics, including reactance. Power control based on a desired power levels the point may thus imply other operational setpoints, such as frequency, to manage operation of the power supply and acoustophoresis device to handle a range of loads.

In some implementations, an RF linear amplifier is used to supply power to the transducer. The linear amplifier may operate by receiving an input signal, which may be AC or DC, and amplifying the input signal in accordance with the operational characteristics of the linear amplifier. Linear amplifiers are typically designed to have a linear response, such that any input signal is amplified by the same gain, within the operating parameters or specifications of the linear amplifier. This linear operation can be achieved through the use of techniques that contribute to linearizing the response of the linear amplifier, potentially in areas where non-ideal conditions tend to impose nonlinearities on the response. However, linear operation is attained at the cost of power regulation, usually generating significant heat losses as well as incurring inefficient operation. Accordingly, linear amplifiers tend to consume significant amounts of power, even when the magnitude of the input signal is relatively small and/or when the gain is relatively small. When demands are placed on the linear amplifier to supply power in response to changing system conditions, such as frequency or loading, challenges are presented in terms of responsiveness and avoiding overloads.

In addition, linear amplifiers are designed for nominal applications, for example, where a 50 ohm load is specified. The load applied to the linear amplifier is thus intended to be composed of mostly real impedance, or resistance, and tolerates a relatively small amount of reactive impedance. In the case of providing power to an acoustic transducer that is composed of a piezoelectric material, the power supply sees a highly reactive load, which limits the usefulness of an RF linear amplifier as the power supply.

Discussed herein is a power supply and method for providing power to an acoustic transducer composed of a piezoelectric material, such as PZT-8. The piezoelectric material may be formed as a poly-crystal, which is also referred to as a crystal herein. The power supply provides RF power with a relatively wide bandwidth of operation to permit responsive operation with relatively high efficiency and with the ability to accommodate a wide range of loads. The PZT driver combines a power converter, such as a buck, buck-boost or boost power converter, with an RF frequency, DC to AC inverter.

The generation of an acoustic standing wave in a fluid medium may be accomplished with the use of an oscillator or function generator and an amplifier. The function generator or oscillator provides an electronic input to a piezoelectric device such that the piezoelectric device vibrates at the frequency that is set by the function generator or oscillator. The amplifier generates a certain amount of power that is provided to the piezoelectric material, which power can determine the strength of the acoustic wave that is set by the frequency of the function generator or oscillator. A controller implementing a control scheme is provided for the amplifier and the function generator or oscillator to control the generated and applied power.

A function generator is utilized to generate the initial wave pattern that is imparted to the acoustic resonator system that includes at least one acoustic transducer that is composed, for example, of a piezoelectric material. The system may include another transducer and/or one or more reflectors that are coupled to an acoustic chamber. The signal from the function generator is controlled for various parameters, such as, for example, amplitude. For example, the signal from the function generator is amplified to increase the amount of power applied to the transducer. The power applied to the transducer determines, at least in part, the power of the acoustic standing wave. The control of power applied to the transducer can thus control the power of the acoustic standing wave. The parameters of the signal from the function generator, such as frequency, amplitude and phase, can be controlled with a controller. The amplification of the signal from the function generator can also be controlled by a controller, which may be the same or different from the function generator controller.

The characteristics of the oscillator input to the piezoelectric material of the acoustic transducer can be modified to permit various vibration modes of the piezoelectric material. For example, a pure sine wave can induce a very succinct vibration of the piezoelectric material, while a signal with harmonic content can cause parasitic vibrations of the piezoelectric material. The input to the piezoelectric material may influence the heat generated or input into the fluid in which the acoustic standing wave is formed. The input may generate more complicated motion in the fluid coupled with the piezoelectric material.

Additionally, driving a piezoelectric material with a current source rather than a voltage source may permit greater electro-mechanical freedom in supporting and sustaining desirable vibratory modes in the piezoelectric material. A drive and control scheme can be provided to generate a low harmonic signal into the piezoelectric material. The control of the acoustic transducer that generates the acoustic standing wave in the fluid medium can utilize a feedback loop and a computational processor. An inductor-capacitor-inductor (LCL) circuit configuration may be used to generate a low harmonic function wave, such as a sine wave, into the piezoelectric material. The low harmonic sine wave permits less parasitic vibrations of the piezoelectric material. Such a sine wave may also permit the piezoelectric material to generate less heat when it vibrates.

The acoustic transducer can be driven to create a multi-dimensional acoustic standing wave in a coupled medium, where the wave has at least non-zero acoustic forces in a direction transverse to the propagation direction of the wave. The multi-dimensional acoustic standing wave generation process takes advantage of the higher-order vibratory modes of a loosely suspended piezoelectric plate.

Piezoelectric material changes shape based on an electrical signal applied to it, such as a voltage or current signal, or based on a corresponding electric field permeating the material. The electric field from external charges affects the fields of the bound charges in the material and thereby affects the shape of the material. The electrical signal can be from a voltage source. In that case the amount of material deformation is related to the voltage applied. For example, the deformation may be ‘voltage clamped’ or ‘voltage damped’. The amount of charge induced is related to the applied voltage and the properties of the material. This relationship can be expressed mathematically as Q=C*V, where Q is charge, C is material capacitance, and V is the voltage of the applied signal. Electrodes may be attached to the piezoelectric material to provide a conduit for the applied signal. In that case the voltage, and the corresponding electric field, is a function of the externally applied charges. Using the above equation, the voltage can be express as V=Q/C. The resultant voltage may be ‘unconstrained’ in relation to operation of the piezoelectric device. The ‘C’ of the piezoelectric device is due to its physical geometry and material properties. Since the material changes shape as a function of the electric field permeating it, the ‘C’ of the device is a function of the electric field permeating it. For a given Q, and driving the material with a current source that is a time varying source of charge, C changes as a function of electric field, which changes the voltage across the device to ‘accommodate’ the changed C. In a voltage driven system, the electric field can determine the amount of charge, which can determine the degree of deformation and correspondingly the amount of change in C. To encourage multimode behavior in piezoelectric material, the piezoelectric material can be configured to be ‘free floating’, and in some examples, is made to be as free floating as possible in both a mechanical and electrical sense.

The control of the multi-dimensional acoustic standing wave and the acoustic resonator or transducer is an important part of an acoustophoresis process. For example, as a multi-dimensional acoustic standing wave is utilized to trap biologic cells and cell debris from a bioreactor process, the reactance of the resonator changes. By sensing the voltage and current of the RF transmission line to the piezoelectric element, the resonator can be properly tuned to optimize the acoustophoresis process. The reactance and power can be extracted from the voltage and current signals on the piezoelectric element. For example, voltage and current signals can be provided to a digital signal processor (DSP), which can be used to calculate RF reactance and power. The measured and calculated parameters of operation for the piezoelectric element can be used to provide feedback for the tuning process. This tuning process may consist of adjusting the gain of the amplifier to achieve a desired power that is provided to the piezoelectric element and/or adjusting the frequency of the drive signal to achieve a desired reactance of the resonator, as examples.

The multi-dimensional acoustic standing wave is generated through a multimode perturbation of the piezoelectric material by electronic signal generated by a function generator or oscillator and modified by an amplifier. The generation of the multi-dimensional acoustic standing wave and the multimode perturbation of the piezoelectric material is described in U.S. Pat. No. 9,228,183 which is incorporated herein by reference.

An RF power driver is provided to drive the acoustic transducer. In some implementations, the power driver is composed of a DC-DC converter coupled to a DC-AC inverter. A filter is provided between the converter and inverter. The output of the inverter may be supplied to the LCL matching filter. The RF power driver has a number of advantages over the linear amplifiers discussed above, including more efficient operation, better responsiveness and the ability to drive highly reactive loads.

The DC-DC converter may be a buck, buck-boost or boost converter, as examples, although any type of DC-DC converter may be used. The amplifier used in conjunction with the function generator or oscillator discussed above can be can be implemented as the converter and filter. The filter can be implemented as an RLC filter with a bandwidth that permits the filter output, such as output voltage, to respond to dynamic changes of the transducer and/or the acoustic cavity.

The RF processing of the linear amplifier discussed above can be synthesized with a DC-DC converter and a DC to AC inverter. The inverter receives a DC input from the converter and provides an RF frequency output. The RF output is controlled by a pulse-width modulated, fixed amplitude pulse train running at the operating frequency of the PZT-cavity system being driven. The amplitude of the output RF signal is controlled by the output of the DC-DC converter. Both the converter and inverter get operational commands from a digital or analog controller. The inverter output can be applied to the LCL matching filter, which smoothes the output of the inverter and provides an impedance match for the output of the inverter to permit efficient electrical power transfer.

A control, which may be a digital or analog control, is provided that can receive inputs fed back from the acoustic transducer or other system components and provide control signals to various components of the RF power converter. The control can provide control signals to vary the DC output of the converter, and/or modify and control the amplitude of the power of the drive signal for the acoustic transducer. Control signals provided by the control can vary the operation of the inverter to modify and control the frequency of the drive signal. The RF power driver with the control permits control and modulation of the acoustic transducer as a highly reactive load, while maintaining desired transducer and acoustic chamber performance.

A control technique provides a system and method for locating desired operating points for an acoustic transducer-cavity combination, with or without loading, which loading may be highly reactive. Feedback from the acoustic transducer can be used to locate the resonance and anti-resonance frequencies of transducer operation. According to some implementations, an operating frequency less than the transducer anti-resonance is inspected for minimum reactance as a point of operation. Some implementations locate a frequency above the anti-resonance frequency, which frequency is inspected for maximum reactance as a point of operation. According to these implementations, a desired level of efficiency can be obtained for acoustophoresis using the acoustic transducer to generate an acoustic standing wave through fluid in the acoustic chamber or cavity to which the transducer is coupled. The points of operation that are determined according to a control technique discussed herein can be frequency setpoints, which can be dynamically maintained. For example, a desired point of operation may change with characteristics of operation of the acoustic chamber, such as a degree of material separation, temperature, power delivered to the transducer, and other phenomena that may influence or modify a desired operating point.

### BRIEF DESCRIPTION OF THE SEVERAL VIEWS OF THE DRAWINGS

The disclosure is described in greater detail below, with reference to the accompanying drawings, in which:

FIG. 1 is a diagram showing an acoustic chamber and connections thereto;

FIG. 2 is a diagram illustrating acoustophoresis with an acoustic transducer and reflector;

FIG. 3 is a cross-sectional side view of an acoustic transducer;

FIG. 4 is a cross-sectional side view of an acoustic transducer with a free piezoelectric element;

FIG. 5 is a cross-sectional view of an acoustic transducer with a damped piezoelectric element;

FIG. 6 is a graph illustrating force applied to a particle in a fluid;

FIG. 7 is a graph illustrating impedance of a piezoelectric element;

FIG. 8A is a diagram illustrating different vibrational modes for an acoustic transducer;

FIG. 8B is an isometric view of an acoustic chamber;

FIG. 8C is a left side elevation view of the acoustic chamber in FIG. 8B;

FIG. 8D is a front elevation view of the acoustic chamber in FIG. 8B;

FIG. 9 is a graph illustrating transducer frequency responses and frequencies with dominant modes;

FIG. 10 is a circuit diagram of an RF power supply with an LCL network;

FIG. 11 is a flowchart illustrating a method for controlling an acoustic transducer;

FIG. 12 is a flowchart illustrating a method for implementing an optimized low pass filter;

FIG. 13 is a graph illustrating a frequency response for an acoustic transducer;

FIG. 14 is a graph illustrating a frequency response for an acoustic transducer;

FIG. 15 is a block diagram illustrating a control technique for an acoustic transducer;

FIG. 16 is a block diagram illustrating a control technique for an acoustic transducer;

FIG. 17 is a block diagram illustrating a calculation technique for obtaining control parameters for an acoustic transducer;

FIG. 18 is a block diagram illustrating demodulation of a voltage or current signal;

FIG. 19 is a flowchart illustrating a control technique for an acoustic transducer;

FIG. 20 is a flowchart illustrating components of a control technique for use with an acoustic transducer;

FIG. 21 is a graph illustrating a frequency response for an LC network;

FIG. 22 is a graph illustrating power, reactance, resistance and peak performance for an acoustic transducer;

FIG. 23 is a graph illustrating a resistance curve versus frequency, with a number of different high-order modes identified;

FIG. 24 is a graph illustrating reactance versus frequency, with a number of different high-order modes identified;

FIGS. 25, 26, 27 and 28 are graphs illustrating turbidity and reactance for a given example of acoustophoresis;

FIG. 29 is a graph illustrating piezoelectric displacement;

FIG. 30 is a graph illustrating power and impedance amplitude;

FIG. 31 is a graph illustrating absolute impedance amplitude;

FIG. 32 is a graph illustrating impedance phase;

FIG. 33 is a graph illustrating displacement normalized by power;

FIG. 34 is a graph illustrating average pressure normalized by power;

FIG. 35 shows two graphs illustrating axial and lateral radiation force;

FIG. 36 shows five graphs illustrating displacement for various modes;

FIGS. 37, 38 are graphs illustrating relationships between dimensions of piezoelectric material and number of modes;

FIG. 39 is a graph illustrating operation with a planar wave at zero phase;

FIG. 40 is a graph illustrating multimode operation at minimum reactance;

FIG. 41 is a graph illustrating resistance, reactance and real power versus frequency;

FIG. 42 is a graph illustrating multimode operation at minimum reactance;

FIGS. 43, 44, 45 and 46 are flowcharts illustrating hardware and software configurations;

FIG. 47 shows graphs illustrating a frequency sweep response;

FIG. 48 is a graph illustrating regions of operation;

FIG. 49 is a graph illustrating an example control technique;

FIGS. 50, 51, 52 and 53 are graphs illustrating various parameters versus frequency.

### DETAILED DESCRIPTION

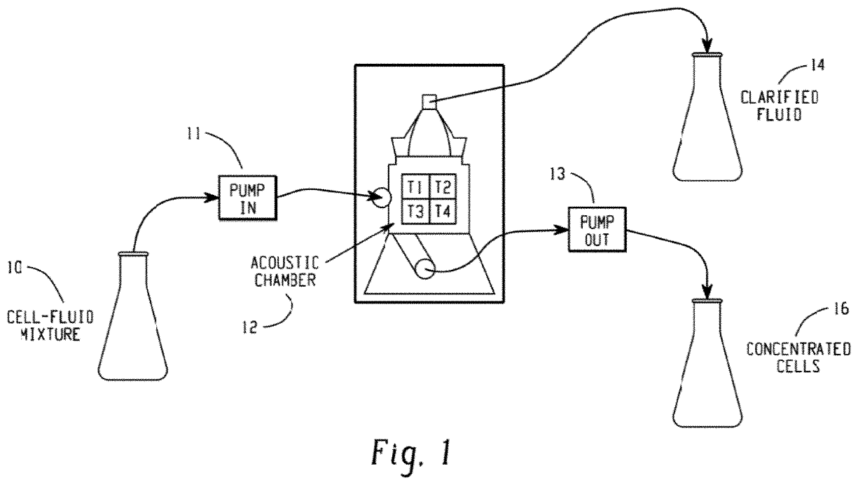

FIG. 1 is a broad overview of an acoustic wave separator system. A mixture 10 of a host fluid and a secondary phase (e.g. particles, cells, or a second different fluid) is sent via a pump 11 into an acoustic chamber 12. Here, the mixture is a cell-fluid mixture. In the acoustic chamber, the secondary phase is concentrated out of the host fluid. The concentrated cells 16 are sent by another pump 13 to be collected. The host fluid, which is more clarified due to the removal of the concentrated cells, is separately collected (indicated by reference numeral 14). Generally speaking, the acoustic chamber has at least one inlet and at least one outlet.

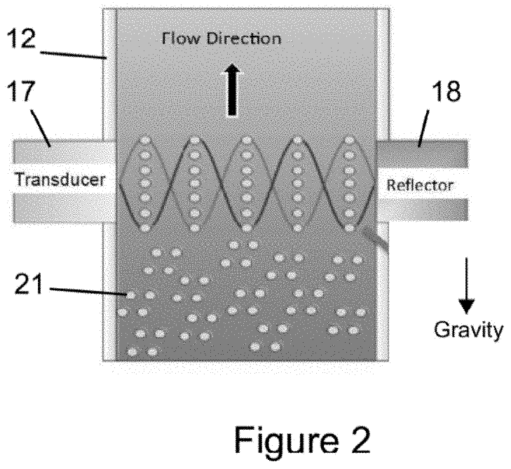

The acoustic chamber operates as shown in FIG. 2. One or more multi-dimensional acoustic standing waves are created between an ultrasonic transducer 17 and a reflector 18. The standing wave is illustrated as beginning and ending with local minima, however, other implementations are possible. For example, the standing wave can be offset at the transducer or the reflector so that local minima or maxima are spaced from the transducer or from the reflector. The reflected wave (or wave generated by an opposing transducer) can be in or out of phase with the transducer generated wave. The characteristics of the standing wave can be modified and/or controlled by the drive signal applied to the transducer, such as by modifying and/or controlling the phase, amplitude or frequency of the drive signal. Acoustically transparent or responsive materials may also be used with the transducer or reflector to modify and/or control the standing wave.

As the fluid mixture flows through acoustic chamber 12 with ultrasonic transducer 17 active, particles or secondary fluid 21 cluster, collect, agglomerate, aggregate, clump, or coalesce at the nodes or anti-nodes of the multi-dimensional acoustic standing wave, depending on the particles' or secondary fluid's acoustic contrast factor relative to the host fluid. The particles form clusters that eventually exit the multi-dimensional acoustic standing wave nodes or anti-nodes when the clusters have grown to a size large enough to overcome the holding force of the multi-dimensional acoustic standing wave (e.g. coalescence or agglomeration overcomes gravity or buoyancy forces). For fluids/particles that are more dense than the host fluid (such as the cells of FIG. 1), the clusters sink to the bottom and can be collected separately from the clarified host fluid. For fluids/particles that are less dense than the host fluid, the buoyant clusters float upwards and can be collected.

The scattering of the acoustic field off the particles results in a three-dimensional acoustic radiation force, which acts as a three-dimensional trapping field. The acoustic radiation force is proportional to the particle volume (e.g. the cube of the radius) when the particle is small relative to the wavelength. The force is proportional to frequency and the acoustic contrast factor. The force scales with acoustic energy (e.g. the square of the acoustic pressure amplitude). When the acoustic radiation force exerted on the particles is stronger than the combined effect of fluid drag force and buoyancy and gravitational force, the particles are trapped within the acoustic standing wave field. The particle trapping in a multi-dimensional acoustic standing wave results in clustering, concentration, agglomeration and/or coalescence of the trapped particles. Relatively large solids of one material can thus be separated from smaller particles of a different material, the same material, and/or the host fluid through enhanced gravitational/buoyancy separation.

The multi-dimensional standing wave generates acoustic radiation forces in both the axial direction (e.g., in the direction of the standing wave, between the transducer and the reflector, which may be at an angle across the flow direction, and in some instances may be perpendicular to the flow direction) and the lateral direction (e.g., in the flow direction or transverse to the direction between the transducer and the reflector). As the mixture flows through the acoustic chamber, particles in suspension experience a strong axial force component in the direction of the standing wave. Since this acoustic force is across (e.g. perpendicular to) the flow direction and the drag force, it quickly moves the particles to pressure nodal planes or anti-nodal planes, depending on the contrast factor of the particle. The lateral acoustic radiation force acts to move the concentrated particles towards the center of each planar node, resulting in clustering, agglomeration or clumping. The lateral acoustic radiation force component can overcome fluid drag for such clumps of particles, to continually grow the clusters, which can exit the mixture due to gravity or buoyancy. The drop in drag per particle as the particle cluster increases in size, as well as the drop in acoustic radiation force per particle as the particle cluster grows in size, may separately or collectively influence operation of the acoustic separator device. In the present disclosure, the lateral force component and the axial force component of the multi-dimensional acoustic standing wave are of the same or different order of magnitude. In this regard, it is noted that in a multi-dimensional acoustic standing wave generated by a single transducer, the axial force is stronger than the lateral force, but the lateral force of such a multi-dimensional acoustic standing wave is much higher than the lateral force of a planar standing wave, usually by two orders of magnitude or more.

Particle drag and acoustic radiation force effects may influence optimal operation of the systems and methods of the present disclosure. At low Reynolds numbers of less than 10, laminar flow dominates, and viscous forces are much stronger than inertial forces.

As the particles are trapped by the multi-dimensional ultrasonic acoustic standing wave, they begin to aggregate and form a clump of particles. The drag on this clump of particles is a function of the geometry of the clump and is not merely the sum of the drag of the individual particles that make up the clump.

For laminar flow, the Navier Stokes equation is expressed as:

$$ρ ( \frac{\partial V}{\partial t} + ( V \cdot ∇ ) V ) ) = - ∇ P + μ {∇}^{2} V$$

where

$$\frac{\partial V}{\partial t}$$

represents unsteady motion, (V·∇)V)represents inertial motion, −∇P represents pressure motion, and μ∇²V represents viscous motion.

For low Reynolds numbers, the unsteady motion and inertial motion terms can be ignored (i.e. set equal to zero), and the equation can be simplified to:

∇P=μ∇²V

For a particle of diameter a, the following equations hold:

$$∇ P ∝ μ \frac{V}{a}$$

$$F = 6 π μ a V$$

where P is pressure, μ is the dynamic viscosity, a is the particle diameter, V is the flow velocity, and F is the Stoke's drag.

Prior to discussing further optimization of the systems, it is helpful to provide an explanation now of how multi-dimensional acoustic standing waves are generated. The multi-dimensional acoustic standing wave used for particle collection is obtained by driving an ultrasonic transducer composed of a piezoelectric material at a frequency that generates the acoustic standing wave and excites a fundamental 3D vibration mode of the transducer. The transducer may be composed of various materials that may be perturbed to generate an ultrasonic wave. For example, the transducer may be composed of a piezoelectric material, including a piezoelectric crystal or poly-crystal. Perturbation of the piezoelectric material, which may be a piezoelectric crystal or poly-crystal, in the ultrasonic transducer to achieve a multimode response allows for generation of a multi-dimensional acoustic standing wave. A piezoelectric material can be specifically designed to deform in a multimode response at designed frequencies, allowing for generation of a multi-dimensional acoustic standing wave. The multi-dimensional acoustic standing wave may be generated with distinct modes of the piezoelectric material such as a 3×3 mode that generates multi-dimensional acoustic standing waves. A multitude of multi-dimensional acoustic standing waves may also be generated by allowing the piezoelectric material to vibrate through many different mode shapes. Thus, the material can be selectively excited to operate in multiple modes such as a 0×0 mode (i.e. a piston mode), 1×1, 2×2, 1×3, 3×1, 3×3, and other higher order modes. The material can be operated to cycle through various modes, in a sequence or skipping past one or more modes, and not necessarily in a same order with each cycle. This switching or dithering of the material between modes allows for various multi-dimensional wave shapes, along with a single piston mode shape to be generated over a designated time.

Some further explanation of the ultrasonic transducers used in the devices, systems, and methods of the present disclosure may be helpful as well. In this regard, the transducers may be composed of a piezoelectric material, such as a piezoelectric crystal or poly-crystal, which may be made of PZT-8 (lead zirconate titanate). Such crystals may have a major dimension on the order of 1 inch and larger. The resonance frequency of the piezoelectric material may nominally be about 2 MHz, and may be operated at one or more frequencies. Each ultrasonic transducer module can have only one crystal, or can have multiple crystals that each act as a separate ultrasonic transducer and are either controlled by one or multiple controllers, which controllers may include signal amplifiers. The piezoelectric material can be square, rectangular, irregular polygon, or generally of any arbitrary shape. The transducer(s) is/are used to create a pressure field that generates forces of the same order of magnitude both orthogonal to the standing wave direction (lateral) and in the standing wave direction (axial).

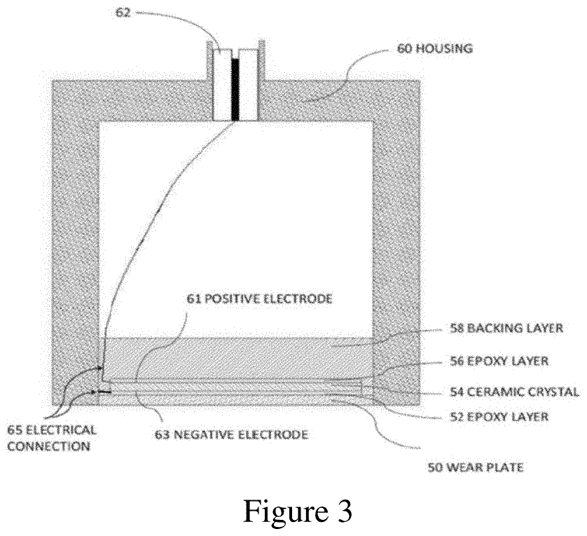

FIG. 3 is a cross-sectional diagram of a conventional ultrasonic transducer. This transducer has a wear plate 50 at a bottom end, epoxy layer 52, ceramic crystal 54 (made of, e.g. PZT), an epoxy layer 56, and a backing layer 58. On either side of the ceramic crystal, there is an electrode: a positive electrode 61 and a negative electrode 63. The epoxy layer 56 attaches backing layer 58 to the crystal 54. The entire assembly is contained in a housing 60 which may be made out of, for example, aluminum. An electrical adapter 62 provides connection for wires to pass through the housing and connect to leads (not shown) which attach to the crystal 54. Typically, backing layers are designed to add damping and to create a broadband transducer with uniform displacement across a wide range of frequency and are designed to suppress excitation at particular vibrational eigen-modes. Wear plates are usually designed as impedance transformers to better match the characteristic impedance of the medium into which the transducer radiates.

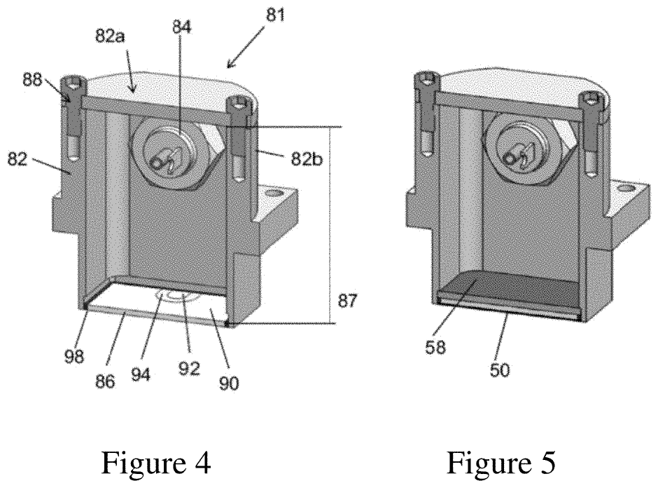

FIG. 4 is a cross-sectional view of an ultrasonic transducer 81 according to an example of the present disclosure. Transducer 81 is shaped as a disc or a plate, and has an aluminum housing 82. The piezoelectric crystal is a mass of perovskite ceramic crystals, each consisting of a small, tetravalent metal ion, usually titanium or zirconium, in a lattice of larger, divalent metal ions, usually lead or barium, and O2− ions. As an example, a PZT (lead zirconate titanate) crystal 86 defines the bottom end of the transducer, and is exposed from the exterior of the housing. The crystal has an interior surface and an exterior surface. The crystal is supported on its perimeter by a small elastic layer 98, e.g. silicone or similar material, located between the crystal and the housing. Put another way, no wear layer is present. In particular embodiments, the crystal is an irregular polygon, and in further embodiments is an asymmetrical irregular polygon.

Screws 88 attach an aluminum top plate 82a of the housing to the body 82b of the housing via threads. The top plate includes a connector 84 for powering the transducer. The top surface of the PZT crystal 86 is connected to a positive electrode 90 and a negative electrode 92, which are separated by an insulating material 94. The electrodes can be made from any conductive material, such as silver or nickel. Electrical power is provided to the PZT crystal 86 through the electrodes on the crystal. Note that the crystal 86 has no backing layer or epoxy layer. Put another way, there is an air gap 87 in the transducer between aluminum top plate 82a and the crystal 86 (i.e. the housing is empty). A minimal backing 58 (on the interior surface) and/or wear plate 50 (on the exterior surface) may be provided in some embodiments, as seen in FIG. 5.

The transducer design can affect performance of the system. A typical transducer is a layered structure with the ceramic crystal bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure the transducers, there is no wear plate or backing, allowing the crystal to vibrate in one of its eigenmodes (i.e. near eigenfrequency) with a high Q-factor. The vibrating ceramic crystal/disk is directly exposed to the fluid flowing through the acoustic chamber.

Removing the backing (e.g. making the crystal air backed) also permits the ceramic crystal to vibrate at higher order modes of vibration with little damping (e.g. higher order modal displacement). In a transducer having a crystal with a backing, the crystal vibrates with a more uniform displacement, like a piston. Removing the backing allows the crystal to vibrate in a non-uniform displacement mode. The higher order the mode shape of the crystal, the more nodal lines the crystal has. The higher order modal displacement of the crystal creates more trapping lines, although the correlation of trapping line to node is not necessarily one to one, and driving the crystal at a higher frequency will not necessarily produce more trapping lines.

In some embodiments, the crystal may have a backing that minimally affects the Q-factor of the crystal (e.g. less than 5%). The backing may be made of a substantially acoustically transparent material such as balsa wood, foam, or cork which allows the crystal to vibrate in a higher order mode shape and maintains a high Q-factor while still providing some mechanical support for the crystal. The backing layer may be a solid, or may be a lattice having holes through the layer, such that the lattice follows the nodes of the vibrating crystal in a particular higher order vibration mode, providing support at node locations while allowing the rest of the crystal to vibrate freely. The goal of the lattice work or acoustically transparent material is to provide support without lowering the Q-factor of the crystal or interfering with the excitation of a particular mode shape.

Placing the crystal in direct contact with the fluid also contributes to the high Q-factor by avoiding the dampening and energy absorption effects of the epoxy layer and the wear plate. Other embodiments may have wear plates or a wear surface to prevent the PZT, which contains lead, from contacting the host fluid. This may be desirable in, for example, biological applications such as separating blood. Such applications might use a wear layer such as chrome, electrolytic nickel, or electroless nickel. Chemical vapor deposition could also be used to apply a layer of poly(p-xylylene) (e.g. Parylene) or other polymers or polymer films. Organic and biocompatible coatings such as silicone or polyurethane are also usable as a wear surface.

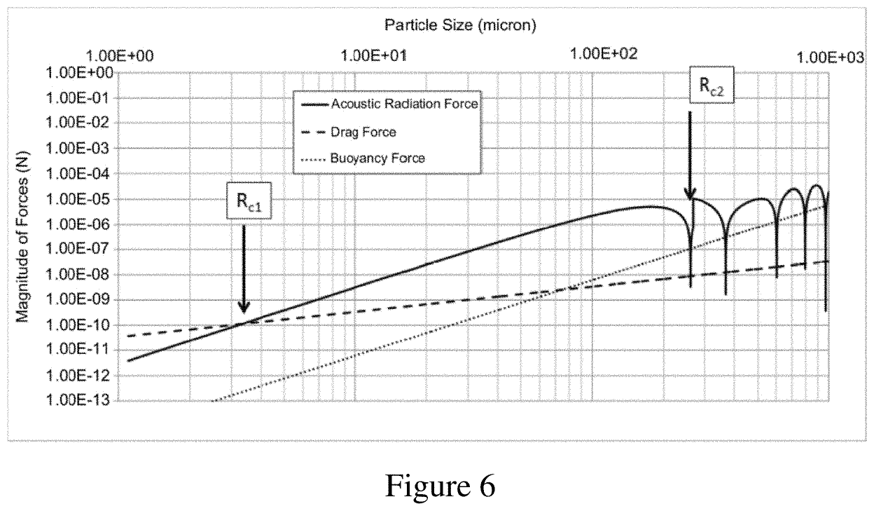

FIG. 6 is a log-log graph (logarithmic y-axis, logarithmic x-axis) that shows the scaling of the acoustic radiation force, fluid drag force, and buoyancy force with particle radius, and provides an explanation for the separation of particles using acoustic radiation forces. The buoyancy force is a particle volume dependent force, and is therefore negligible for particle sizes on the order of micron, but grows, and becomes significant for particle sizes on the order of hundreds of microns. The fluid drag force (Stokes drag force) scales linearly with fluid velocity, and therefore typically exceeds the buoyancy force for micron sized particles, but is negligible for larger sized particles on the order of hundreds of microns. The acoustic radiation force scaling is different. When the particle size is small, Gor'kov's equation is accurate and the acoustic trapping force scales with the volume of the particle. Eventually, when the particle size grows, the acoustic radiation force no longer increases with the cube of the particle radius, and will rapidly vanish at a certain critical particle size. For further increases of particle size, the radiation force increases again in magnitude but with opposite phase (not shown in the graph). This pattern repeats for increasing particle sizes.

Initially, when a suspension is flowing through the system with primarily small micron sized particles, the acoustic radiation force balances the combined effect of fluid drag force and buoyancy force to permit a particle to be trapped in the standing wave. In FIG. 6 this trapping happens at a particle size labeled as R_{c1}. The graph then indicates that all larger particles will be trapped as well. Therefore, when small particles are trapped in the standing wave, particle clustering/coalescence/clumping/aggregation/agglomeration takes place, resulting in continuous growth of effective particle size. As particles cluster, the total drag on the cluster is much lower than the sum of the drag forces on the individual particles. In essence, as the particles cluster, they shield each other from the fluid flow and reduce the overall drag of the cluster. As the particle cluster size grows, the acoustic radiation force reflects off the cluster, such that the net acoustic radiation force decreases per unit volume. The acoustic lateral forces on the particles may be larger than the drag forces for the clusters to remain stationary and grow in size.

Particle size growth continues until the buoyancy force becomes dominant, which is indicated by a second critical particle size, R_{c2}. The buoyancy force per unit volume of the cluster remains constant with cluster size, since it is a function of the particle density, cluster concentration and gravity constant. Therefore, as the cluster size increases, the buoyancy force on the cluster increases faster than the acoustic radiation force. At the size R_{c2}, the particles will rise or sink, depending on their relative density with respect to the host fluid. At this size, acoustic forces are secondary, gravity/buoyancy forces become dominant, and the particles naturally drop out or rise out of the host fluid. Some particles may remain in the acoustic wave as clusters as others drop out, and those remaining particles and new particles entering the acoustic chamber with the flow of a fluid mixture continue to move to the three-dimensional nodal locations, repeating the growth and drop-out process. As clusters grow to around the size of the wavelength, the acoustic force on the cluster drops off rapidly. As clusters grow in sized to greater than a wavelength, the acoustic force rises rapidly. This phenomenon of rapidly decreasing and increasing acoustic force is shown at and above size R_{c2}, to the right in the graph in FIG. 6 with the periodic sharp changes in acoustic radiation force on the cluster. This periodic phenomenon is attributed to a cluster size of one or multiple wavelengths. Thus, FIG. 6 explains how small particles can be trapped continuously in a standing wave, grow into larger particles or clumps, and eventually rise or settle out because of increased buoyancy/gravity force.

In some examples, the size, shape, and thickness of the transducer can determine the transducer displacement at different frequencies of excitation. Transducer displacement with different frequencies may affect particle separation efficiency. Higher order modal displacements can generate three-dimensional acoustic standing waves with strong gradients in the acoustic field in all directions, thereby creating strong acoustic radiation forces in all directions, which forces may, for example be equal in magnitude, leading to multiple trapping lines, where the number of trapping lines correlate with the particular mode shape of the transducer.

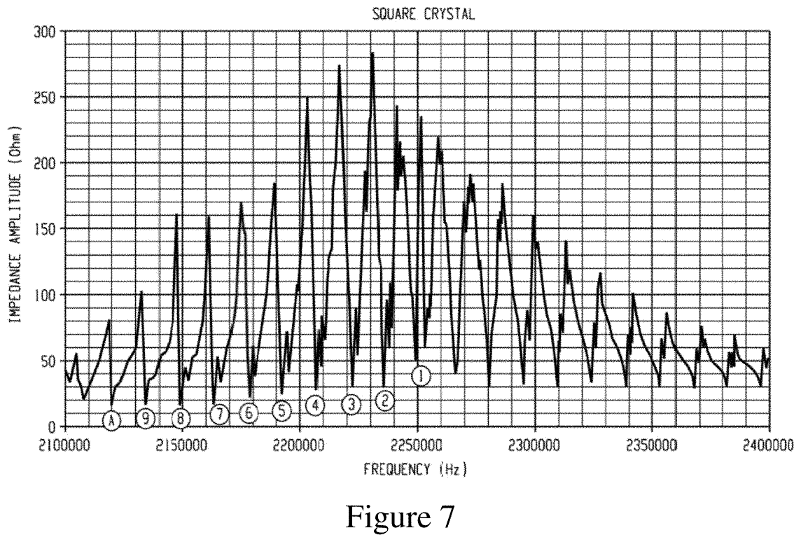

FIG. 7 shows the measured electrical impedance amplitude of the transducer as a function of frequency in the vicinity of the 2.2 MHz transducer resonance. The minima in the transducer electrical impedance correspond to acoustic resonances of a water column and represent potential frequencies for operation. Numerical modeling has indicated that the transducer displacement profile varies significantly at these acoustic resonance frequencies, and thereby directly affects the acoustic standing wave and resulting trapping force. Since the transducer operates near its thickness resonance, the displacements of the electrode surfaces are essentially out of phase. The typical displacement of the transducer electrodes may not be uniform and varies depending on frequency of excitation. Higher order transducer displacement patterns result in higher trapping forces and multiple stable trapping lines for the captured particles.

To investigate the effect of the transducer displacement profile on acoustic trapping force and particle separation efficiencies, an experiment was repeated ten times, with all conditions identical except for the excitation frequency. Ten consecutive acoustic resonance frequencies, indicated by circled numbers 1-9 and letter A on FIG. 7, were used as excitation frequencies. The conditions were experiment duration of 30 min, a 1000 ppm oil concentration of approximately 5-micron SAE-30 oil droplets, a flow rate of 500 ml/min, and an applied power of 20 W.

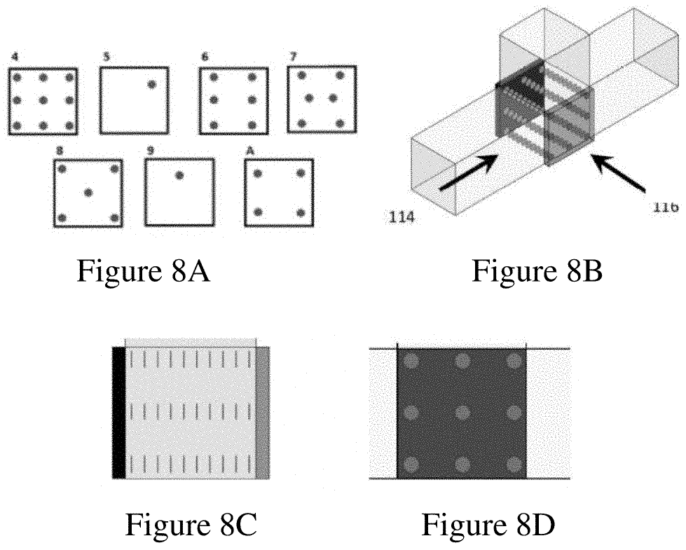

As the emulsion passed by the transducer, the trapping lines of oil droplets were observed and characterized. The characterization involved the observation and pattern of the number of trapping lines across the fluid channel, as shown in FIG. 8A, for seven of the ten resonance frequencies identified in FIG. 7.

FIG. 8B shows an isometric view of the system in which the trapping line locations are being determined. FIG. 8C is a view of the system as it appears when looking down the inlet, along arrow 114. FIG. 8D is a view of the system as it appears when looking directly at the transducer face, along arrow 116.

The effect of excitation frequency clearly determines the number of trapping lines, which vary from a single trapping line at the excitation frequency of acoustic resonance 5 and 9, to nine trapping lines for acoustic resonance frequency 4. At other excitation frequencies four or five trapping lines are observed. Different displacement profiles of the transducer can produce different (more) trapping lines in the standing waves, with more gradients in displacement profile generally creating higher trapping forces and more trapping lines. It is noted that although the different trapping line profiles shown in FIG. 8A were obtained at the frequencies shown in FIG. 7, these trapping line profiles can also be obtained at different frequencies.

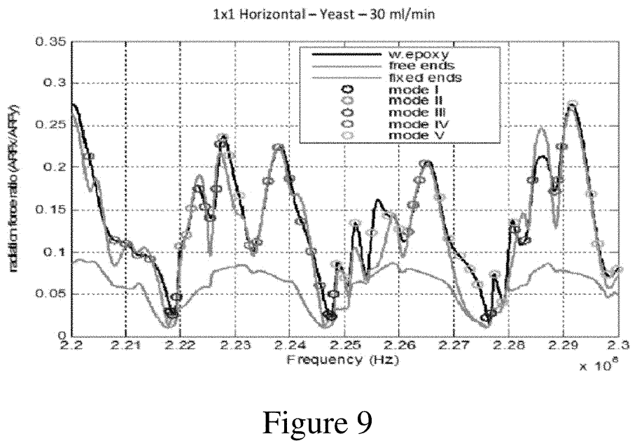

FIG. 8A shows the different crystal vibration modes possible by driving the crystal to vibrate at different fundamental frequencies of vibration. The 3D mode of vibration of the crystal is carried by the acoustic standing wave across the fluid in the chamber all the way to the reflector and back. The resulting multi-dimensional standing wave can be thought of as containing two components. The first component is a planar out-of-plane motion component (uniform displacement across crystal surface) of the crystal that generates a standing wave, and the second component is a displacement amplitude variation with peaks and valleys occurring in lateral directions across the crystal surface. Three-dimensional force gradients are generated by the standing wave. These three-dimensional force gradients result in lateral radiation forces that stop and trap the particles with respect to the flow by overcoming the viscous drag force. In addition, the lateral radiation forces are responsible for creating tightly packed clusters of particles. Therefore, particle separation and gravity-driven collection depends on generating a multi-dimensional standing wave that can overcome the particle drag force as the mixture flows through the acoustic standing wave. Multiple particle clusters are formed along trapping lines in the axial direction of the standing wave, as presented schematically in FIG. 8A. The piezoelectric crystals of the transducers described herein can be operated at various modes of response by changing the drive parameters, including frequency, for exciting the crystal. Each operation point has a theoretically infinite number of vibration modes superimposed, where one or more modes are dominant. In practice, multiple vibration modes are present at arbitrary operating points of the transducer, with some modes dominating at a given operating point. FIG. 9 presents COMSOL results for crystal vibration and lateral radiation forces on a typical particle size. The ratio of lateral to axial radiation force is plotted versus operating frequency. Points are labeled on the curve where a specific mode of vibration is dominant. Mode I represents the planar vibration mode of the crystal designed to generate a 2 MHz standing wave in a mixture. Mode III represents the 3×3 mode operation of a 1×1 crystal. These analytical results show that the 3×3 mode can be dominant with different levels of lateral radiation force. More specifically, operating the example system at a frequency of 2.283 MHz generates the lowest lateral force ratio of about 1.11 for a 3×3 mode. This operating point generates the largest cluster size and the best collection operation for the example system. Operating the devices and systems described herein at a frequency for a given configuration that produces a desired 3D mode with the lowest lateral force ratio is desirable to achieve the most efficient separation.

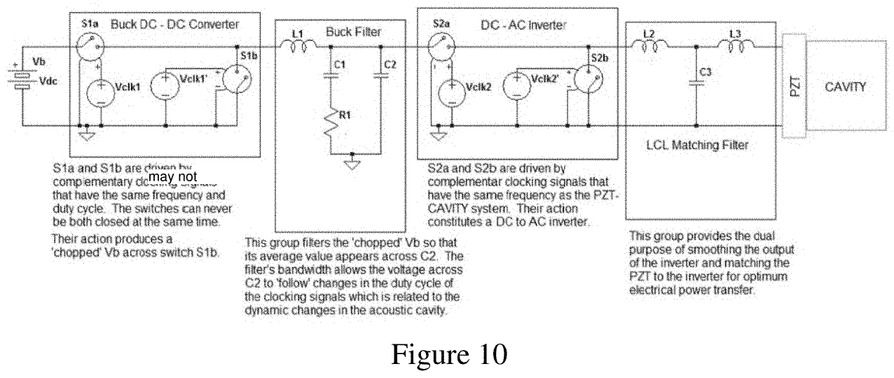

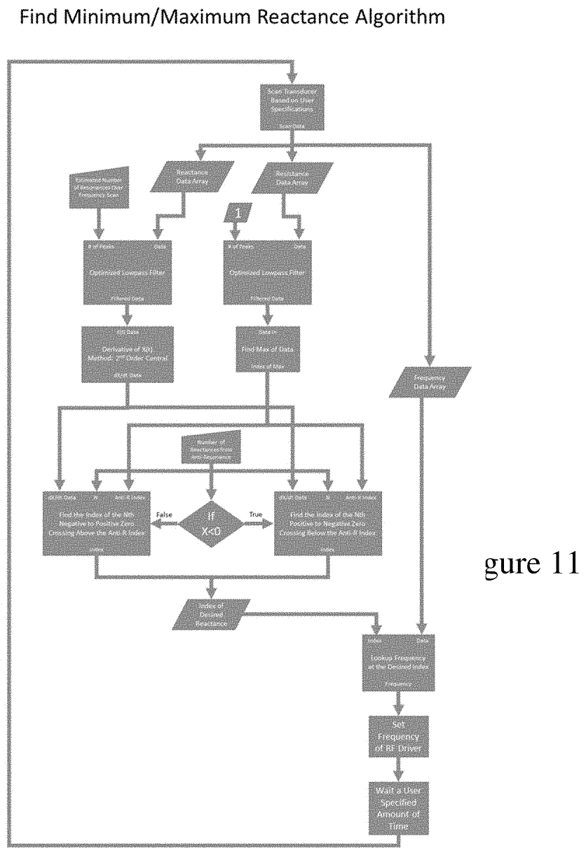

FIG. 10 is a diagram of an RF power converter composed of a DC-DC converter, a converter filter, a DC-AC inverter and an LCL matching filter. The switches of the converter are driven by complementary clocking signals that have the same frequency and duty cycle. The switches may be operated to avoid being both closed at the same time. The output of the converter is a chopped signal with an average DC voltage that is dependent on the duty cycle of the switches.

The output of the converter is provided to an RLC filter that averages the output of the converter. The chopped output of the converter appears as an average DC signal across the output of the filter. The filter's bandwidth or response is sufficient to follow or keep up with changes in the duty cycle of the clocking signals provided to the switches of the converter. The duty cycle of the clocking signals, or the DC output of the converter, is related to control of the dynamic characteristics of the acoustic transducer, for example, the reactive nature of the piezoelectric material.

The output of the filter is provided to the DC-AC inverter. The inverter includes switches that are driven by complementary clocking signals that are switched at a frequency that is related to the operation of the acoustic transducer and cavity system. The DC input to the inverter is used as a control signal for RF power conversion, where the inverter provides an RF signal with a power level that is controlled by the DC input.

The output of the inverter is applied to an LCL matching filter, which is connected to the acoustic transducer. The LCL matching filter smoothes the output of the inverter and provides a load match for the inverter output.

Referring to FIG. 11, a flow chart is illustrated for a process for locating a minimum and/or maximum reactance for the acoustic transducer and/or the transducer/acoustic chamber combination, which may be under load. The load can be a fluid in the acoustic chamber, and/or particulates or a secondary fluid that is separated from the primary or host fluid. As the particulates or secondary fluid is separated from the primary or host fluid, the characteristics of the fluid in the acoustic chamber change, which can impact the operation of the transducer and/or transducer/acoustic chamber combination. The process for locating an operating point for driving the transducer begins by scanning through frequencies applied to the transducer, for example, by applying a range of frequencies to the transducer and measuring feedback data from the transducer. The range of frequencies to be scanned can be provided by user settings. Data for the reactance, X, and resistance, R, of the transducer is collected. One technique for collecting reactance and resistance data is to measure voltage, current and phase angle on the transducer. Resistance is determined as the real part of the voltage divided by the current, while reactance is determined as imaginary part of the voltage divided by the current.

As the data for the frequency scan is collected, a number of resonance and anti-resonance frequencies can be determined. The data can be passed through a low pass filter and peaks can be identified using a derivative function. A maximum peak for the anti-resonance is also identified. The method can accept an input setting of the number of reactances from anti-resonance to locate a minimum reactance. Based on the collected and calculated data, the desired minimum reactance below anti-resonance or desired maximum reactance above anti-resonance is determined, in this case as an index of the minimum or maximum reactances. Once the frequency of the desired reactance is located, the frequency of the RF power converter is set to the located frequency. The located frequency can be an operating setpoint for operating the transducer.

After a period of time, such as a number of milliseconds up to a number of tens of seconds, the process is repeated. By repeating the process, variations in the system can be dynamically identified, such as changes to reactance caused by temperature shifts, and the desired operating setpoints can be modified accordingly in keeping with the process.

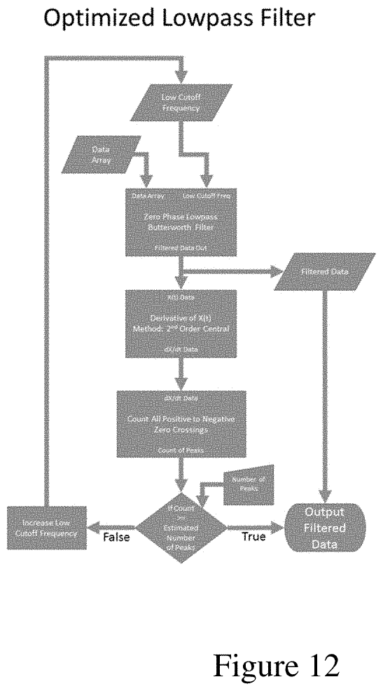

Referring to FIG. 12, a flow chart illustrates a process for implementing a low-pass filter for use in the frequency determination process described above. The filter characteristics can be modified in accordance with the illustrated process to contribute to optimizing detection of the desired frequency setpoints. The process begins by using an existing cut off or corner frequency in conjunction with the data collected from the frequency scan. A zero phase low-pass Butterworth filter is used to filter the collected data with the cutoff frequency. The derivative of the data is taken to determine minimums and/or maximums, and positive to negative zero crossings are identified and counted. The positive to negative zero crossings are indicative of detected peaks in the frequency response. If the process detects more peaks than expected, the cutoff frequency is increased and the process is repeated. If the count is less than the expected number of peaks, the filtered data is provided to the minimum/maximum reactance detection process.

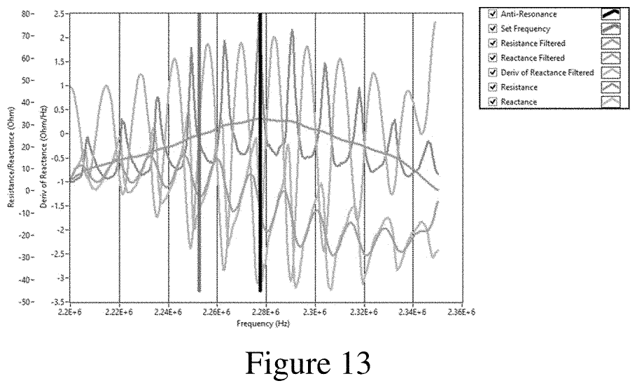

FIG. 13 illustrates a frequency scan for a slightly damped 1×3 piezoelectric transducer coupled to an acoustic cavity through which a fluid containing CHO (Chinese hamster ovary) cells was flowed. As illustrated, peak anti-resonance is located, and a minimum reactance two away from the anti-resonance is selected for a frequency setpoint. In the figure, anti-resonance is approximately 2.278 MHz, and the selected frequency setpoint is approximately 2.251 MHz.

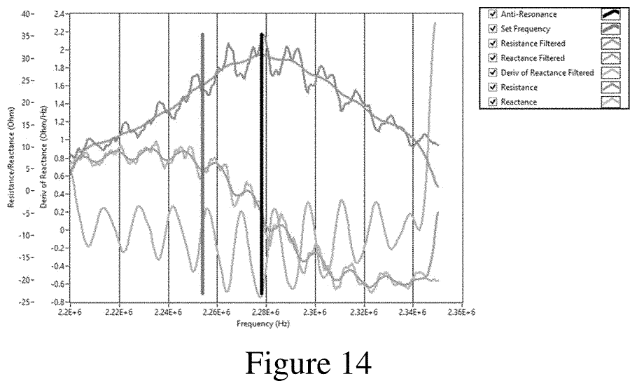

FIG. 14 illustrates a frequency scan for a highly damped 2 MHz 1×3 transducer coupled to an acoustic chamber containing CHO. The peak anti-resonance is identified and the minimum reactance two away from the anti-resonance frequency is selected for an operating setpoint. Although a minimum reactance two away from the anti-resonance frequency is chosen as an operating setpoint, any reactance or index away from anti-resonance can be chosen for an operating setpoint.

Through experimental testing of the large scale acoustic filtration system, it has been determined that the 1 MHz and 2 MHz 1×3 transducer may have an optimal efficiency when operating at the minimum reactance points at frequencies below the transducer anti-resonances, as well as operating at the maximum reactance points above the anti-resonance of the transducer. The technique described herein provides an automated method to set the frequency of the RF drive to the transducer, so it is operating at a minimum reactance point below the anti-resonance or a maximum reactance above the anti-resonance. According to a feature, the technique maintains the desired operating point. The technique can be used to set the frequency of the RF drive, such as the inverter, function generator or oscillator discussed above.

TABLE 1
Functions and Variable Inputs and Outputs

| Name | Type | Description |
|---|---|---|
| Scan | Function | Steps through a range of frequencies and |
| Function | | captures Resistance and Reactance data |
| | | from the Voltage and Current |
| | | measurements of the RF drive. |
| | | Inputs: |
| | | Range (+−50 kHz around anti-res) |
| | | Step Size (500 Hz) |
| | | Step Interval (1 ms) |
| | | Output: |
| | | Array of Frequency, R, and X |
| Estimated | Input | Expected number of resonances over the |
| Number of | Double | full scan range |
| Resonances | | |
| Number of | Input | If negative the method will pick the |
| Reactance | Signed | frequency of that many minima below the |
| Minima/Maxima | Integer | anti-resonance. If positive the method will |
| from Anti- | | pick the frequency of that many maxima |
| Resonance | | above the anti-resonance |
| Frequency | Output | The frequency that the method picks to set |
| to Set | Double | the RF drive |
| Wait Time | Input | Specifies the amount of time between |
| | Double | scans |

The method begins by running a sweep of frequencies and collecting resistance and reactance data for each frequency step. The resistance and reactance data is extrapolated from the voltage and current measurements of the RF drive. The sweep range is specified by the user, but is targeted to be 50 kHz above and 50 kHz below the anti-resonance of the transducer. The step size and step interval are also variables that can be altered. When the sweep is complete it outputs the frequency, resistance, and reactance at each step.

The data from the sweep is then filtered utilizing a zero-phase low pass Butterworth filter. The reactance enters a loop where the low cutoff frequency of the filter is constantly increased, until the number of peaks of the filtered data, equals the number of estimated peaks. This number of estimated peaks is entered by the user. The resistance data is filtered using a zero-phase low-pass Butterworth filter, however the low cutoff frequency is increased until there is one peak. The peak value of the filtered resistance data is interpreted as the anti-resonance of the transducer.

The derivative of the filtered reactance data is calculated and is used to find all the maximum or minimum points of the reactance curve. If the number of reactance minima/maxima from the anti-resonance data input is negative the method will look for the minimum reactance points below the anti-resonance. The method does this by identifying the negative to positive zero crossings, in other words, the upward slope zero crossings of the derivative of the filtered reactance curve. If this number is positive the method will look for the positive to negative zero crossings above the anti-resonance, which are the maximum points of the reactance curve. The absolute value of the number of reactance minima/maxima from the anti-resonance data input is the number of minimum or maximum points from the anti-resonance. The index of this point is used to determine the frequency to set the RF drive.

The RF drive is set and the method waits for a designated amount of time set by the user. Once this time period has elapsed the method then scans and start the sequence over again. Sample data of both slightly and highly damped data can be seen in FIGS. 13 and 14. In both these examples the method was selected to pick two minimum reactance points below the anti-resonance. The set frequency is indicated by the red line. It can be seen that this line falls on the negative to positive zero crossing of the derivative of the filtered reactance data curve, and at the local minimum of the filtered reactance data curve.

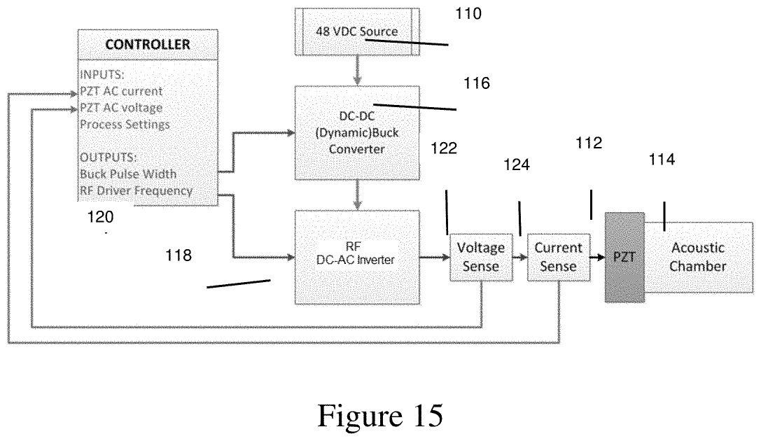

Referring to FIG. 15, a diagram of a control configuration for controlling an acoustic transducer 112 coupled to an acoustic chamber 114 is illustrated. Acoustic transducer 112 is driven by an RF power converter composed of DC source 110, DC-DC converter 116 and RF DC-AC inverter 118. The output drive signal provided by inverter 118 is inspected or sensed to obtain voltage sense 122 and current sense 124, which are fed back to a controller 120. Controller 120 provides control signals to converter 116 and inverter 118 to modulate the drive signal provided to the acoustic transducer 112.

The signal provided by controller 120 to converter 116 is a pulse width measure, which determines the duty cycle of the switching signals in converter 116. The duty cycle determines the DC level of the output of converter 116, which is applied to inverter 118. For example, the greater the duty cycle, the higher the DC output that is generated by converter 116. An example of such a converter is illustrated in FIG. 10. Controller 120 also provides control signals to inverter 118 that determine the frequency of operation of inverter 118. The control signals provided to inverter 118 may be switching signals, for switching switches in inverter 118, an example of such switches being shown in FIG. 10. Alternately, or in addition, controller 120 can provide a control signal to inverter 118 that is used to indicate a desired switching frequency, and circuitry internal to inverter 118 interprets the control signal and switches the internal switches in accordance with the interpreted control signal.

Voltage sense 122 and current sense 124 produce signals that are provided to controller 120 as feedback signals to control the drive signal provided to acoustic transducer 112. Controller 120 performs operations and calculations on the signals provided by voltage sense 122 and current sense 124, for example, to obtain a power measure, P=V*I, or to obtain a phase angle, θ=arctan (X/R).

Controller 120 is provisioned with a control scheme that accepts process settings, such as power output, range of frequency operation, or other user selectable parameters, and provides control signals to converter 116 and inverter 118 based on the process settings and the feedback values. For example, as described above, controller 120 can sequence through a number of frequencies in a range of frequencies that are provided to inverter 118 to scan through the frequency range and determine the characteristics of transducer 112 or transducer 112 in combination with acoustic chamber 114, which may be under load. The results of the frequency scan in terms of voltage and current obtained from the voltage sense 122 and current sense 124, respectively, are used to identify characteristics of the impedance curves for the components or the system, such as is illustrated in FIG. 13. The frequency scan can be implemented to occur at set up, and/or at intervals during operation of the illustrated system. During steady-state operation, the frequency scanned can be conducted to identify desired setpoints for operation, such as power or frequency, based on user settings and feedback values. The control scheme implemented by controller 120 is thus dynamic, and responds to changing conditions in the system, such as may be encountered with frequency drift, temperature change, load changes and any other system parameter changes. The dynamic nature of the control scheme permits the controller to respond to or compensate for nonlinearities, such as may be encountered as components age or lose tolerance. Accordingly, the control scheme is adaptive and can accommodate system changes.

Some examples of system operation include driving acoustic transducer 112 to produce a multidimensional acoustic standing wave in the acoustic chamber 114. A 3D acoustic wave is stimulated by driving acoustic transducer 112, which may be implemented as a piezoelectric crystal, sometimes referred to herein as a PZT, near its anti-resonance frequency. Cavity resonances modulate the impedance profile of the PZT as well as affect its resonance modes. Under the influence of the 3D acoustic field, suspended particles in the liquid medium in the acoustic cavity 114 are forced into agglomerated sheets and then into strings of ‘beads’ of agglomerated material. Once particle concentrations reach a critical size, gravitational forces take over and the agglomerated material drops out of the acoustic field and to the bottom of the chamber. The changing concentrations of agglomerated material as well as the dropping out of that material affects the cavity's resonances which in turn change the acoustic loading on the PZT and its corresponding electrical impedance. The changing dynamics of the collected material detunes the cavity and PZT reducing the effects of the 3D wave in clarifying the medium. Additionally, changes in the medium and cavity temperature also detune the cavity so that clarification is reduced. To track the resonance changes occurring in the cavity, a control technique is used to follow changes in the PZT's electrical characteristics.

A strong 3D acoustic field can be generated by driving the PZT at a frequency where its input impedance is a complex (real and imaginary) quantity. However, cavity dynamics can cause that impedance value to change significantly in an erratic manner. The changes in impedance are due, at least in part, to changes in the load applied to the acoustic transducer 112 and/or acoustic chamber 114. As particles or secondary fluid is separated from a primary or host fluid, the loading on acoustic transducer 112 and/or acoustic chamber 114 changes, which in turn can influence the impedance of the acoustic transducer 112 and/or acoustic chamber 114.

To correct for detuning, controller 120 calculates the PZT impedance from the voltage and current sensed at the PZT using voltage sense 122 and current sense 124 and determines which way to change the operating frequency to compensate for the detuning. Since frequency changes affect power delivered to the chamber, the controller also determines how to adjust the output voltage of (dynamic) buck converter 116 to maintain the desired amount of power output from RF DC-AC inverter 118 and into the acoustic transducer 112 and/or acoustic chamber 114.

Buck converter 116 is an electronically adjustable DC-DC power supply and is the power source for inverter 118. RF DC-AC inverter 118 converts the DC voltage out of converter 116 back to a high-frequency, AC signal to drive the PZT. The dynamics in the chamber occur at rates corresponding to frequencies in the low audio band. Consequently, the converter 116, controller 120, and DC-AC inverter 118 are capable of working at rates faster than the low audio band to permit controller 120 to track chamber dynamics and keep the system in tune.

Controller 120 can simultaneously change the frequency of DC-AC inverter 118 and the DC voltage coming out of buck converter 116 to track cavity dynamics in real time. The control bandwidth of the system is a function of the RF bandwidth of inverter 118 and the cutoff frequency of the filtering system of buck converter 116.

Controller 120 can be implemented as a DSP (digital signal processor) control, or as an FPGA (field programmable gate array) control, as examples. Controller 120 may be implemented with two channels, to permit parallel processing, for example to analyze real and/or reactive impedance, voltage, current and power.

The acoustic dynamics of the cavity affects the electrical characteristics of the PZT which affects the voltage and current drawn the PZT. The sensed PZT voltage and current is processed by the controller to compute the real-time power consumed by the PZT as well as its instantaneous impedance (affected by acoustic dynamics). Based on user set points the controller adjusts, in real-time, the DC power supplied to inverter 118 and the frequency at which inverter 118 is operated to track cavity dynamics and maintain user set points. An LCL network is used to match the output impedance of inverter 118 to increase power transfer efficiency.

Controller 120 samples sensor signals fast enough to detect changes in cavity performance (via changes in PZT impedance) in real time. For example, controller 120 may sample the feedback values from the voltage sense 122 and current sense 124 at one hundred million samples per second. Signal processing techniques are implemented to permit a wide dynamic range for system operation to accommodate wide variations in cavity dynamics and applications. Converter 116 can be configured to have a fast response time to follow the signal commands coming from controller 120. Inverter 118 can drive a wide range of loads that demand varying amounts of real and reactive power that change over time. The electronics package used to implement the system illustrated in FIG. 15 may be configured to meet or exceed UL and CE requirements for electromagnetic interference (EMI).

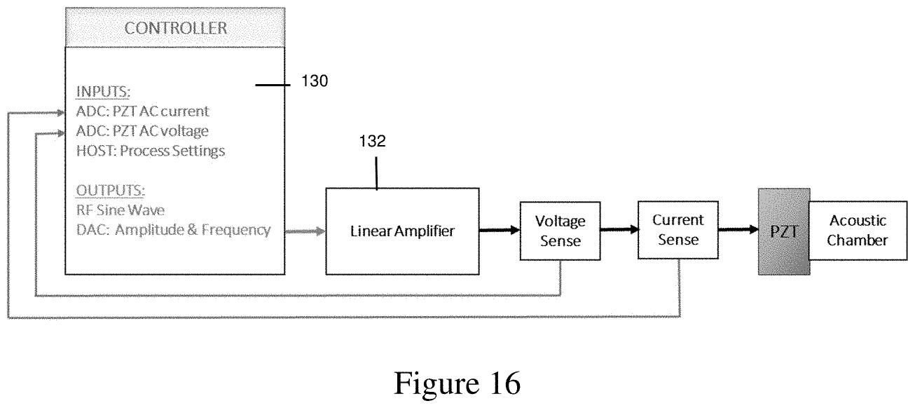

Referring to FIG. 16, controller 120 may be implemented with very-high-speed parallel digital-signal-processing loops using RTL (Register Transfer Level) which is realized in actual digital electronic circuits inside a field-programmable-gate-array (FPGA). Two high speed digital proportional integral (PI) loops adjust the frequency and amplitude control signals generated by controller 120 to track power and reactance. A linear amplifier 132 is used to amplify the output signal from controller 130 (which can be implemented as controller 120) in preparation for driving the PZT. The voltage and current sense is used to sense the voltage and current at the transducer. A calculation is performed in series by controller 130 to generate control signals provided to linear amplifier 132. The FPGA can be operated with a clocking signal of 100 MHz. The clocking speed contributes to obtaining fast enough sampling to monitor and adapt to conditions of the PZT in real-time. In addition, the structure of the FPGA permits each gate component to have a propagation delay commensurate with the clocking speed. The propagation delay for each gate component can be less than one cycle, or 10 ns with a clocking speed of 100 MHz.

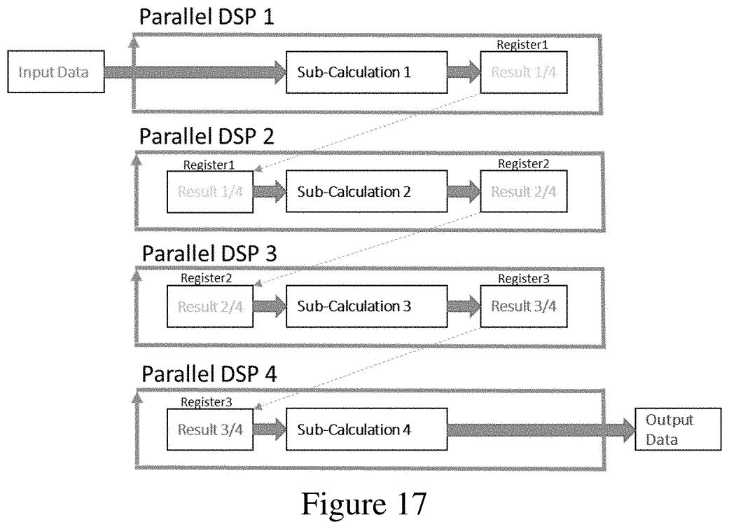

Referring to FIG. 17, a diagram illustrates parallel and sequential operations for calculating control signals. Controller 130 may be configured to calculate the following parameters.

VRMS=sqrt(V1²+V2²+ . . . +Vn²)

IRMS=sqrt(I1²+I2²+ . . . +In²)

Real Power (P=V-Inst.×I-Inst Integrated over N Cycles)

Apparent Power (S=VRMS×IRMS)

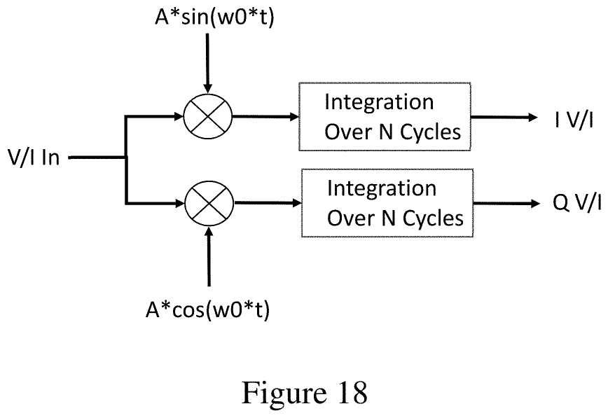

Controller 130 may be configured to calculate reactive power and bipolar phase angle by decomposing sensed voltage and current into in-phase and quadrature-phase components. FIG. 18 illustrates the in-phase and quadrature-phase demodulation of the voltage and current to obtain a four-quadrant phase, reactive power and reactance. The calculations for reactive power and phase angle can be simplified using the in-phase and quadrature-phase components.

VPhase Angle=Arctan(QV/IV)

IPhase Angle=Arctan(QI/II)

Phase Angle=VPhase−Iphase

Reactive Power=(Q=Apparent Power×Sine(Phase Angle)

Controller 130 may implement a control scheme that begins with a frequency sweep to determine system performance parameters at discrete frequencies within the frequency sweep range. The control scheme may accept inputs of a start frequency, a frequency step size and number of steps, which defines the frequency sweep range. Controller 130 provides control signals to linear amplifier 132 to modulate the frequency applied to the PZT, and the voltage and current of the PZT are measured using the voltage sense and the current sense. The control scheme of controller 130 may repeat the frequency sweep a number of times to determine the system characteristics, for example, reactance, with a relatively high level of assurance.

A number of reactance minimums can be identified as a result of analysis of the data obtained in the frequency sweep. The control technique can be provided with an input that specifies a certain frequency range where a desired reactance minimum is located, as well as being provided with a resistance slope (+/−) that can be used for tracking a desired point of operation based on resistance tracking that corresponds to a desired minimum reactance. The resistance slope may be constant near the minimum reactance, which may provide a useful parameter for use with a tracking technique. By tracking resistance at a desired frequency, a robust control can be attained for operating at a minimum reactance point.

The control technique may take the derivative of the resistance/reactance values to locate zero slope derivatives, which are indicative of maximums and minimums. A proportional-integral-differential (PID) controller loop may be used to track the resistance to obtain a frequency setpoint at which a desired minimum reactance occurs. In some implementations, the control may be a proportional-integral (PI) loop. With the FPGA operating at 100 MHz, adjustments or frequency corrections can be made every 10 ns to compensate for changes in the tracked resistance. This type of control can be very accurate and implemented in real-time to manage control of the PZT in the presence of a number of changing variables, including reactance, load and temperature, for examples. The control technique can be provided with an error limit for the frequency of the reactance minimum or frequency setpoint, to permit the control to adjust the output to linear amplifier 132 to maintain the frequency within the error limit.

A fluid mixture, such as a mixture of fluid and particulates, may be flowed through the acoustic chamber to be separated. The fluid mixture flow may be provided via a fluid pump, which may impose perturbations on the fluid, as well as the PZT and chamber. The perturbations can create a significant fluctuation in sensed voltage and current amplitudes, indicating that the effective impedance of the chamber fluctuates with pump perturbations. However, owing to the speed of the control technique, the fluctuations can be almost completely canceled out by the control method. For example, the perturbations can be identified in the feedback data from the PZT and can be compensated for in the control output from the controller. The feedback data, for example the sensed voltage and current, may be used to track the overall acoustic chamber pressure. As the characteristics of the transducer and/or acoustic chamber change over time and with various environmental parameters, such as pressure or temperature, the changes can be sensed and the control technique can compensate for the changes to continue to operate the transducer and acoustic chamber at a desired setpoint. Thus, a desired setpoint for operation can be maintained with very high accuracy and precision, which can lead to optimized efficiency for operation of the system.

The FPGA may be implemented as a standalone module and maybe coupled with a class-D driver. Each module may be provided with a hardcoded address so that it can be identified when connected to a system. The module can be configured to be hot-swappable, so that continuous operation of the system is permitted. The module may be calibrated to a particular system and a transducer, or may be configured to perform a calibration at particular points, such as upon initialization. The module may include long-term memory, such as an EEPROM, to permit storage of time in operation, health, error logs and other information associated with operation of the module. The module is configured to accept updates, so that new control techniques can be implemented with the same equipment, for example.

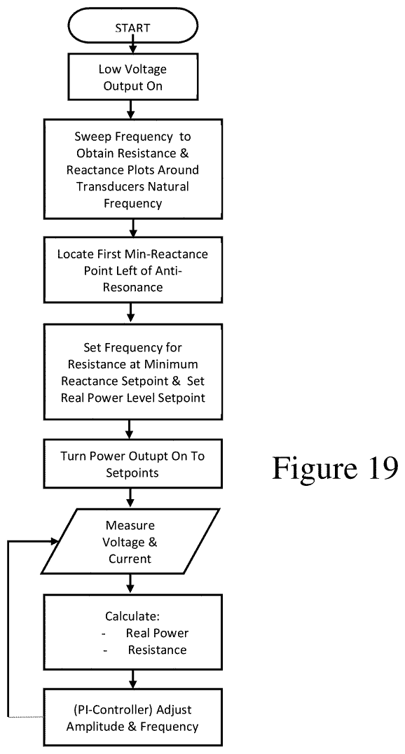

Referring now to FIG. 19, a method for controlling an acoustic transducer is illustrated with a flowchart. The illustrated method may be implemented on or with controller 120 or 130. The method uses a low voltage output during a frequency sweep that drives the acoustic transducer over a range of frequencies. Feedback from the acoustic transducer is used to determine the resistance and reactance response of the transducer over the range of frequencies at the low voltage output. Once the data for the transducer responses collected, the frequency at which the minimum reactance occurs below anti-resonance is identified. The resistance at the minimum reactance is identified and the frequency setpoint is set to establish operation at this resistance. A real power setpoint for the frequency setpoint is established, which may be based on user input. The establishment of the operating setpoints, the method causes the power control signals to be output for the linear amplifier or the converter-inverter power supply.

The method performs a loop in which voltage and current are measured at the acoustic transducer, real power and resistance are calculated and provided to a proportional-integral (PI) controller. The output of the PI controller is used to adjust the amplitude and frequency of the signal supplied to the transducer. The loop is repeated, resulting in the amplitude of the power provided to the transducer being controlled and tracked, and the frequency of the power provided to the transducer being controlled and tracked. The loop permits the controller to dynamically adjust to changes in the system, including changes related to loading of the transducer and/or the transducer/acoustic cavity combination or changes related to temperature, as examples.

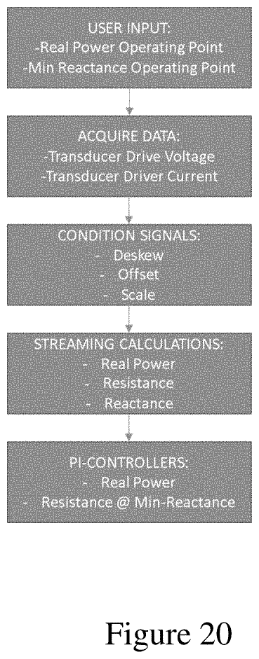

FIG. 20 illustrates an example method for processing information to implement a transducer control. The method uses desired operating points for real power and a minimum reactance, which may be obtained from user input. Data is received from the transducer, including drive voltage and drive current. The data received from the transducer is conditioned to improve the quality of the information and calculations derived there from. For example, the data representing drive voltage and drive current is deskewed, provided with an offset and scaled for use with subsequent calculations. The condition data is used to calculate real power, resistance and reactance of the transducer. These parameters are compared to operating points received in the method, and a PI controller is used to generate a signal that can adjust the real power and frequency of the drive signal provided to the transducer. Note that the conditioned feedback parameters can be used to generate an error signal in conjunction with the desired operating point information, with the error signal being provided to an amplifier that adjusts the signal provided to the power supply, whether linear amplifier or converter-inverter combination.

An LCL matching filter is discussed above, such as with respect to FIG. 15. According to another example, and LC matching filter is provided between the converter output and the PZT. The LC matching filter provides impedance scaling to obtain inappropriate load for the inverter drive. The LC combination can be considered a network, which is tuned to provide desired power transfer, such as optimized power transfer, through the transducer and into the resonant cavity. Considerations for implementing the LCL filter or the LC filter include the combined response of the transducer and the resonant cavity. According to one example, a filter is implemented to permit desired power transfer, such as optimized power transfer, when the acoustic transducer is operated in a multi-dimensional mode, or in a multi-mode, for example, with multiple overlaid vibrational modes that produce one or more primary or dominant vibrational modes. As discussed above, a desired mode of operation is at a frequency that corresponds to a minimum reactance point of the response of the transducer, and/or the response of the transducer/resonant cavity combination.

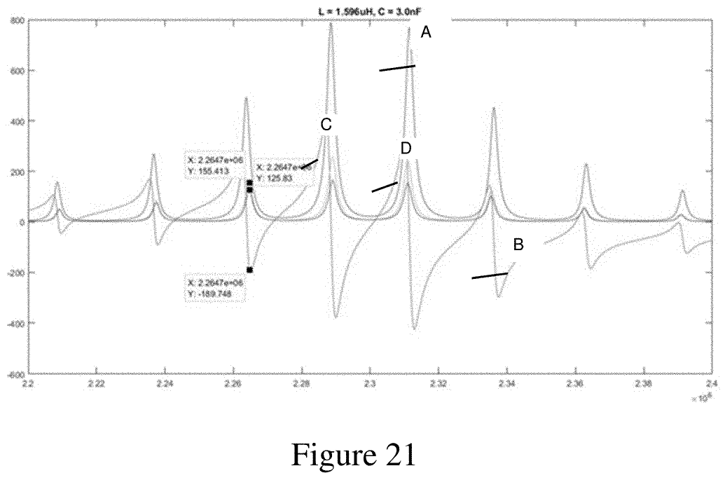

For a fixed resonant frequency, the LC network can deliver different amounts of power based on the system resonances in accordance with the combination of inductor and capacitor values that are used to form the LC network. FIG. 21 illustrates a response curve for an LC network with an inductor value of 1.596 uH and a capacitor value of 3.0 nF. The resonant frequency of the LC network is 2.3 MHz, the resistive impedance (A) is shown in blue, the reactive impedance (B) is shown in red, the input real power (C) is shown in yellow and the acoustic real power (D) into the cavity is shown in purple. With regard to the power delivered into the system, increasing the capacitor value with the same resonance increases power into the system. In general, changing the values of the inductor and/or capacitor can influence the resonant frequency of the LC network. Changing the resonant frequency of the LC network changes the frequency at which optimum power transfer occurs, and can impact the efficiency of the transfer. For example, the frequency for optimum power transfer relative to minimum reactance points (B) of the input impedance of the system is influenced by the resonance frequency of the LC network.

The plot in FIG. 21 shows the points on the input real power (C) and the acoustic real power (D) at a reactance minimum. The input real power and acoustic real power are fairly well matched, indicating efficient transfer of power. If the value of the inductor is changed to 0.8 uH and the value of the capacitor is changed to 6.0 nF, the same reactance minimum produces a greater power transfer with somewhat less efficiency. The power transfer becomes less efficient when the input real power (C) is significantly different (greater) than the acoustic real power (D). In some instances, depending on the inductor and capacitor values, power transfer can be highly efficient, however, the frequency operating point may not be at a minimum reactance point (B). Accordingly, trade of choices can be made between operating the transducer to obtain highly efficient separation in the acoustic chamber, implying a minimum reactance point, and obtaining efficient power transfer into the chamber. For a given material being separated and a given transducer, an LC network can be selected with a resonance frequency to obtain efficient power transfer into the acoustic cavity, improving overall system efficiency.

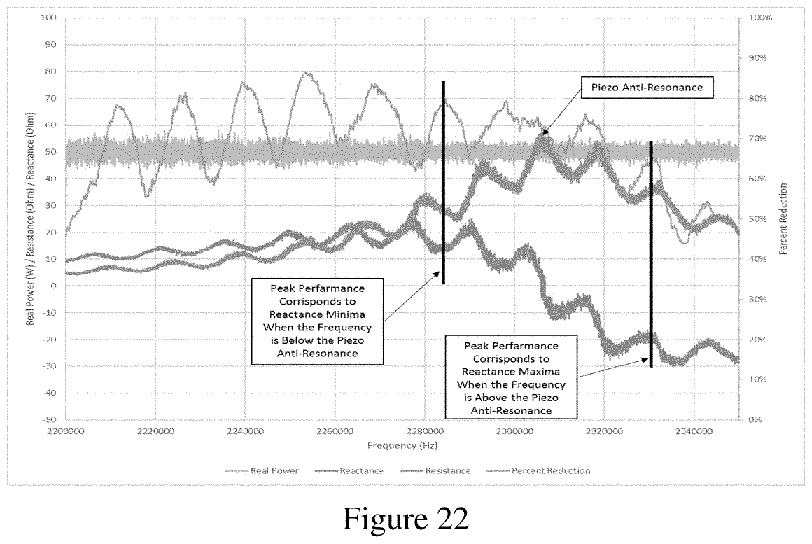

FIG. 22 is a graph of a frequency response for real power/resistance/reactance for an acoustic transducer in an acoustic resonant cavity. The peak performance modes are identified on either side of the transducer anti-resonance. The two different peak performance modes are for different materials in the acoustic cavity. The peak performance to the left of the anti-resonance corresponds to a local minima for reactance, while the peak performance to the right of anti-resonance corresponds to a local maxima for reactance.

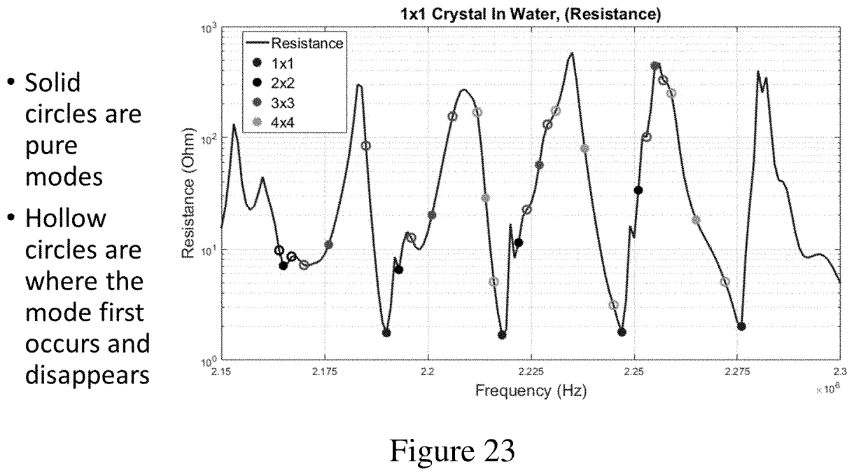

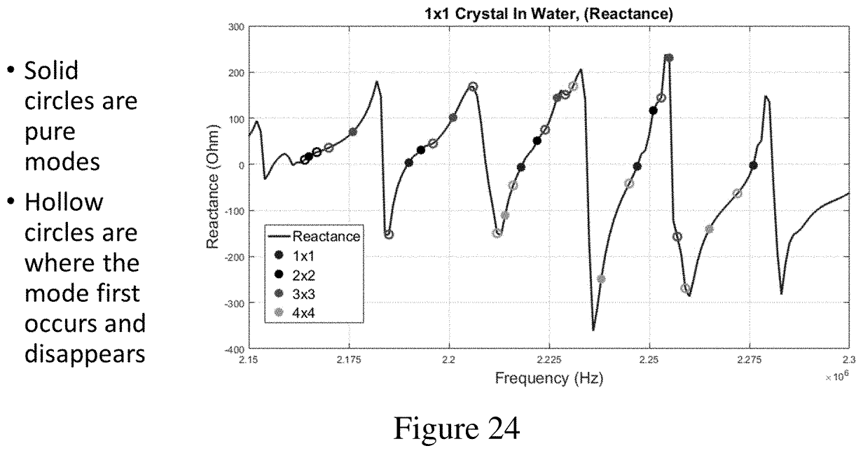

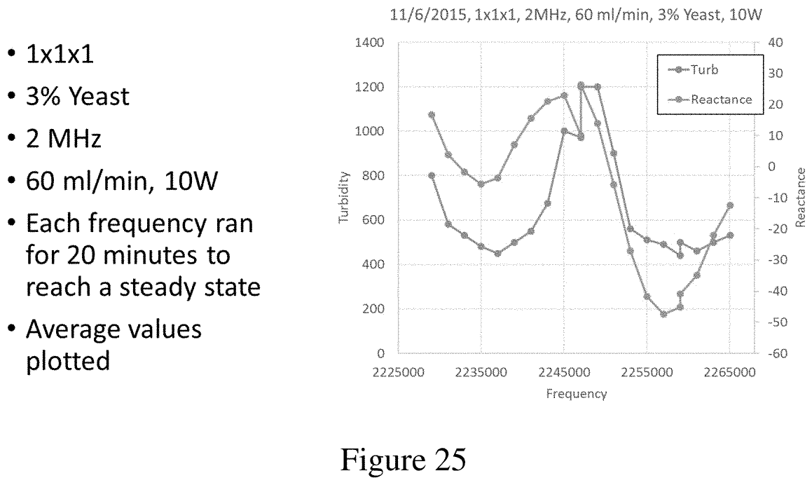

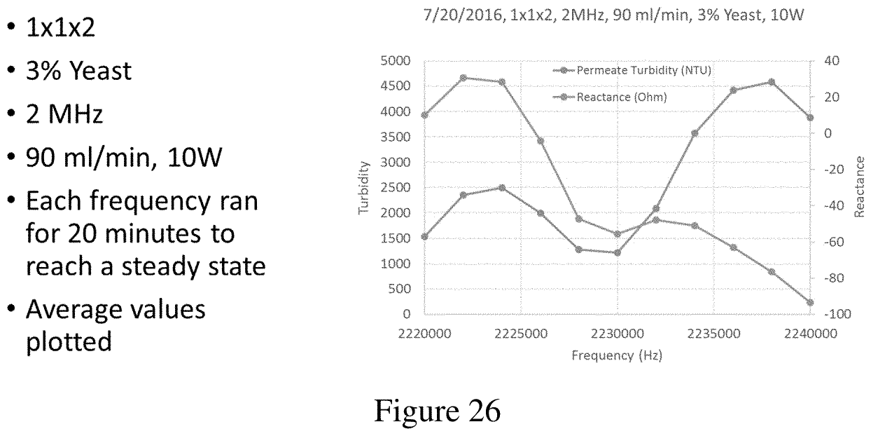

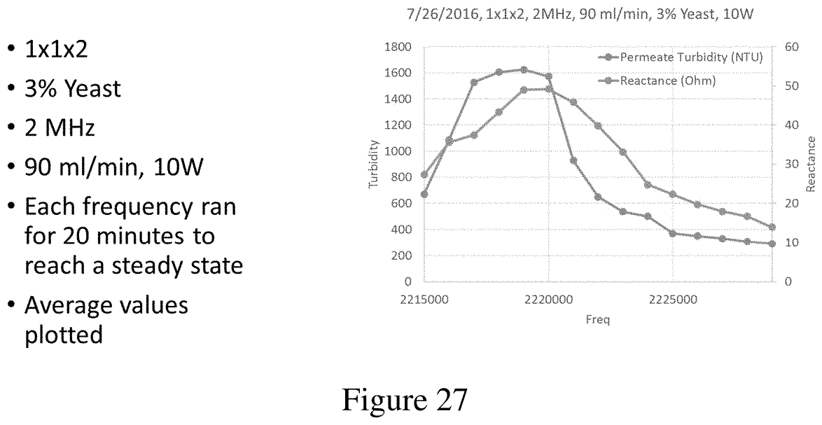

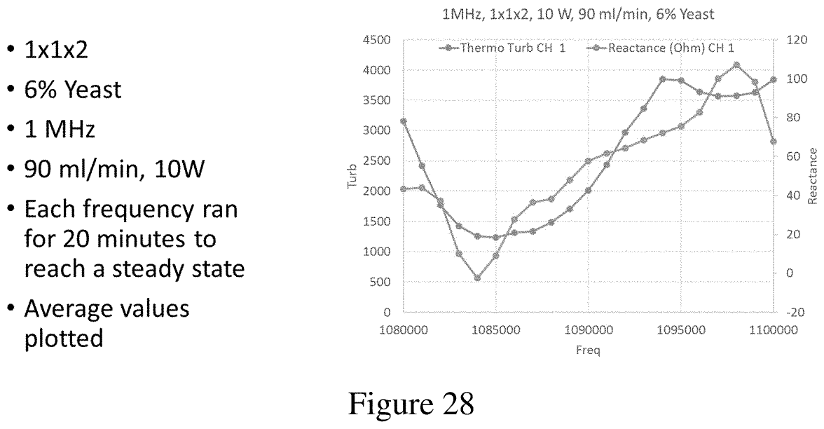

FIG. 23 is a graph illustrating a resistance curve versus frequency, with a number of different higher-order modes of operation identified. Higher order modes are obtained along the graph line locations where resistance is above a minimum. FIG. 24 is a graph illustrating reactance versus frequency, with a number of different higher-order modes identified. Higher order modes are illustrated as available along a number of locations on the graph line. FIGS. 25, 26, 27 and 28 are graphs illustrating turbidity and reactance for a given example of acoustophoresis. The acoustic transducer in FIG. 28 was operated at 1 MHz.

The acoustic radiation force exerted on the particles in the fluid can be calculated and/or modeled. For example, a COMSOL model was created and used to predict linear acoustic standing wave fields. The model implemented models for piezo-electricity, elasticity and acoustics. The model was used to predict acoustic radiation forces on particles that are small compared to wavelength, which includes using the Gorkov equation, and larger particles, which includes using the Yurii-Zhenia equations. In some instances, it may be helpful to normalized the results, for example, by normalizing with respect to power. The effect on the particles of the acoustic radiation forces can be studied, and in particular used for determining transducer configurations, and for controlling the transducer and/or transducer/cavity combination.

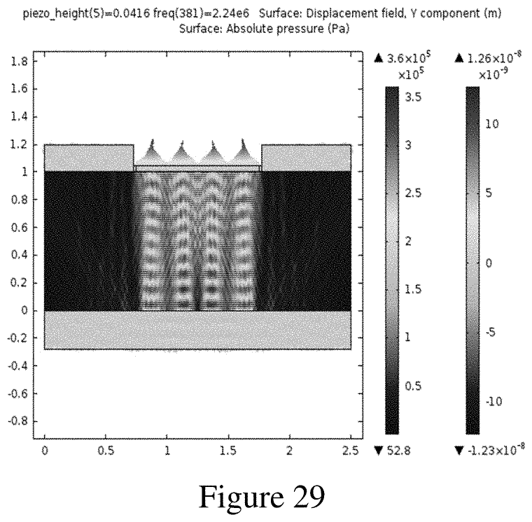

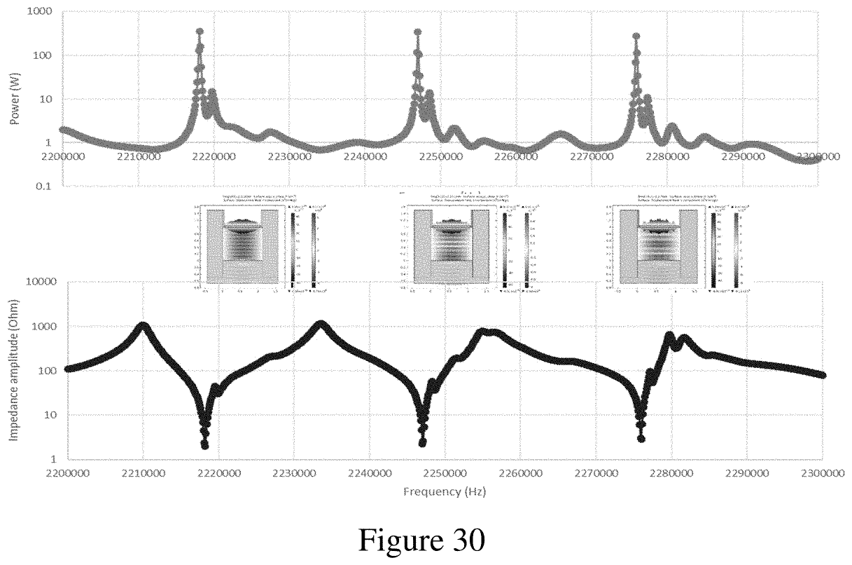

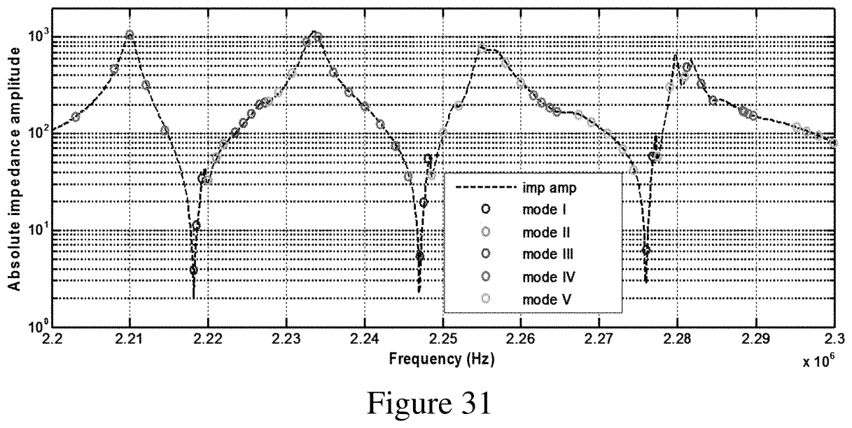

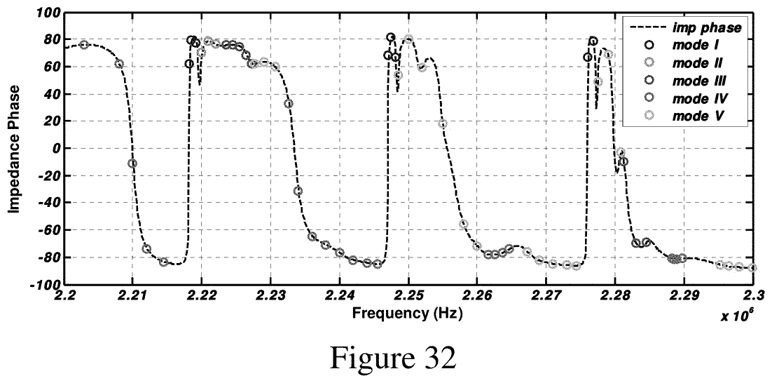

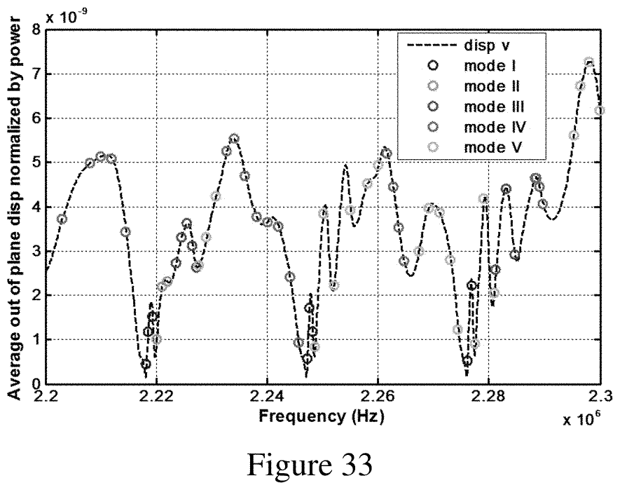

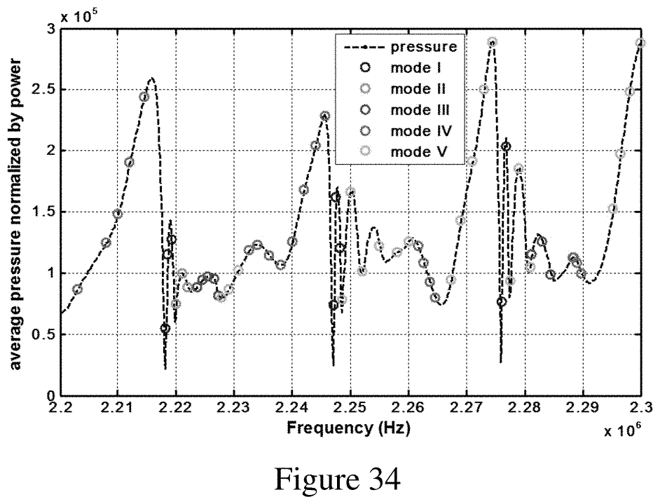

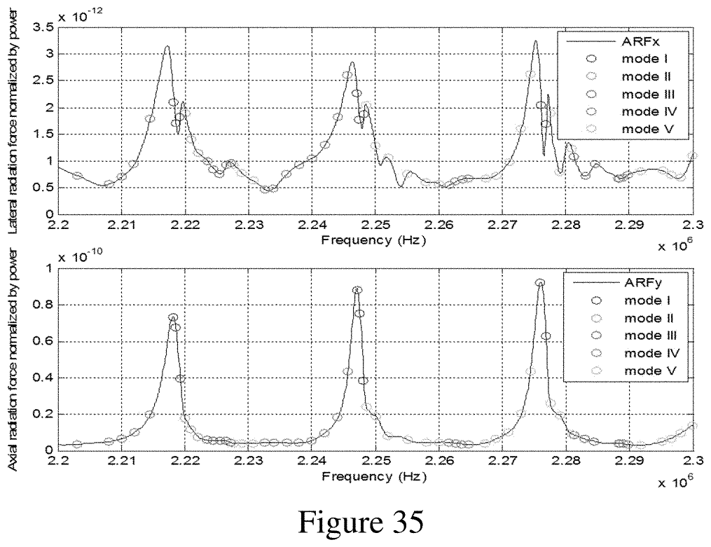

FIG. 29 is a graph illustrating piezoelectric displacement. FIG. 30 is a graph illustrating power and impedance amplitude. FIG. 31 is a graph illustrating absolute impedance amplitude. A number of modes are identified along the line of the graph. Higher order modes can be attained near peak absolute impedance amplitudes. FIG. 32 is a graph illustrating impedance phase. Again, a number of modes are illustrated along the line of the graph. FIG. 33 is a graph illustrating displacement normalized by power. Again, a higher order multimode operation can be attained at higher displacement values. FIG. 34 is a graph illustrating average pressure normalized by power. FIG. 35 shows two graphs illustrating axial and lateral radiation force.

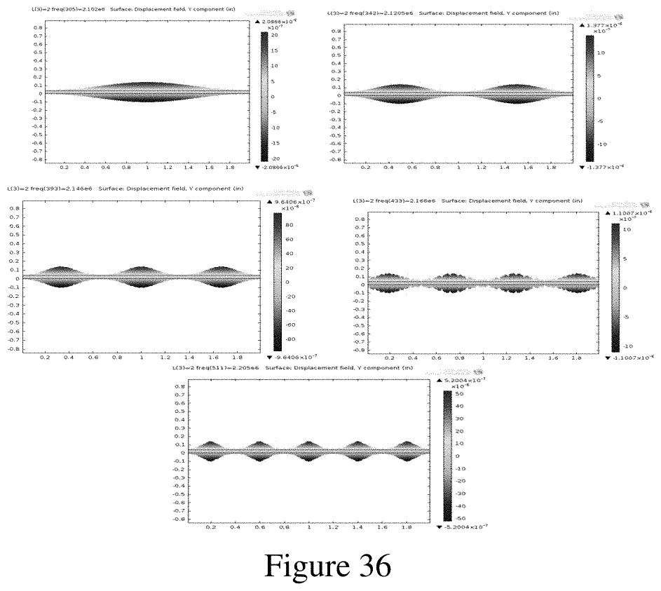

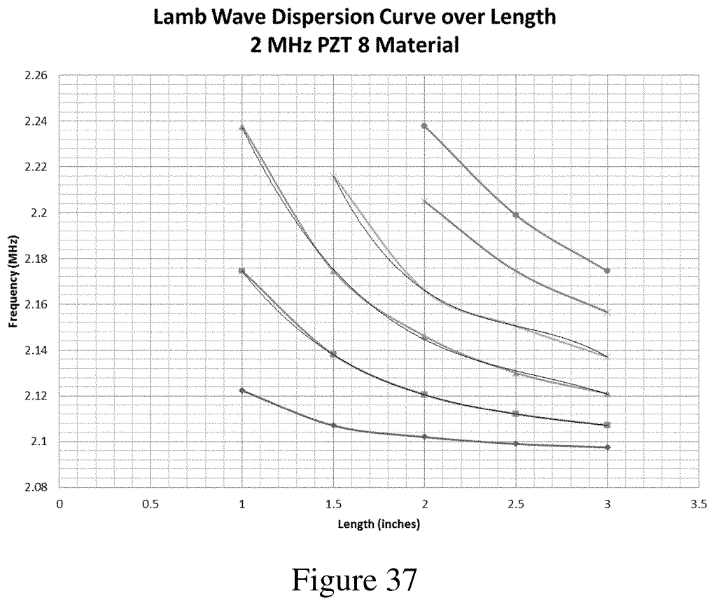

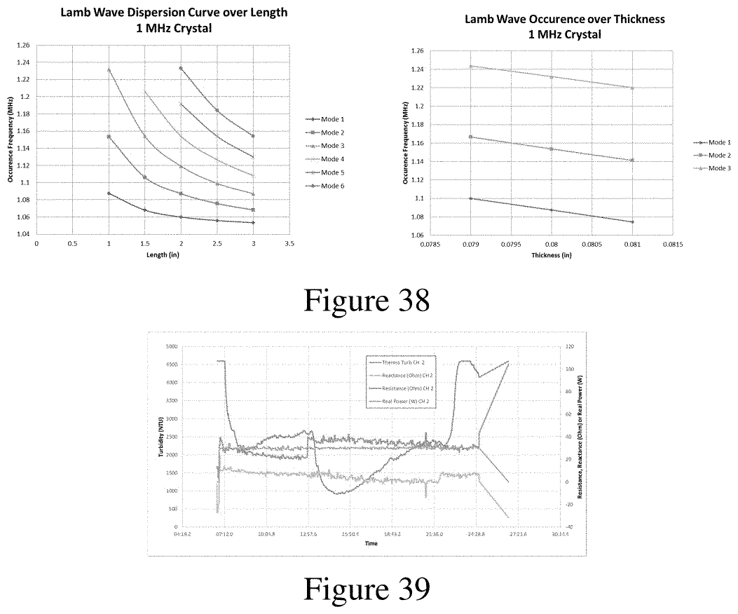

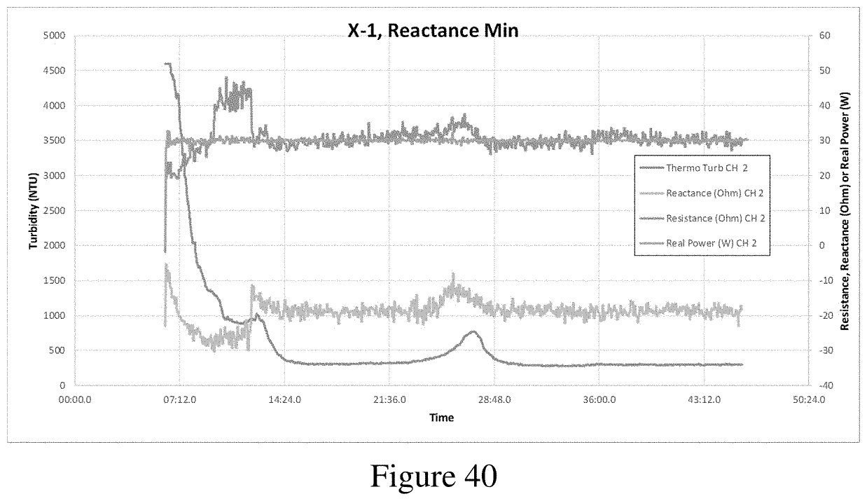

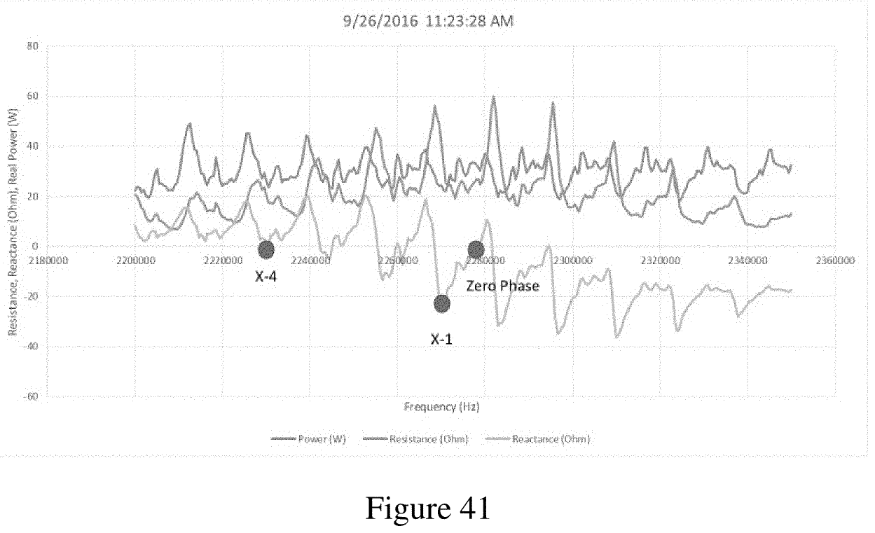

FIG. 36 shows five graphs illustrating displacement for various modes. FIGS. 37, 38 are graphs illustrating relationships between dimensions of piezoelectric material and number of modes. FIG. 39 is a graph illustrating operation with a planar wave at zero phase. FIG. 40 is a graph illustrating multimode operation at minimum reactance. FIG. 41 is a graph illustrating resistance, reactance and real power versus frequency. The performance illustrated in FIG. 39 is fairly poor, with a minimum turbidity of approximately 1000, and typical turbidity performance being much higher. The performance illustrated in FIG. 40 is for operation as illustrated in FIG. 41 and zero phase. The acoustic transducer in this case is producing a planar mode acoustic standing wave, which can be envisioned as piston operation.

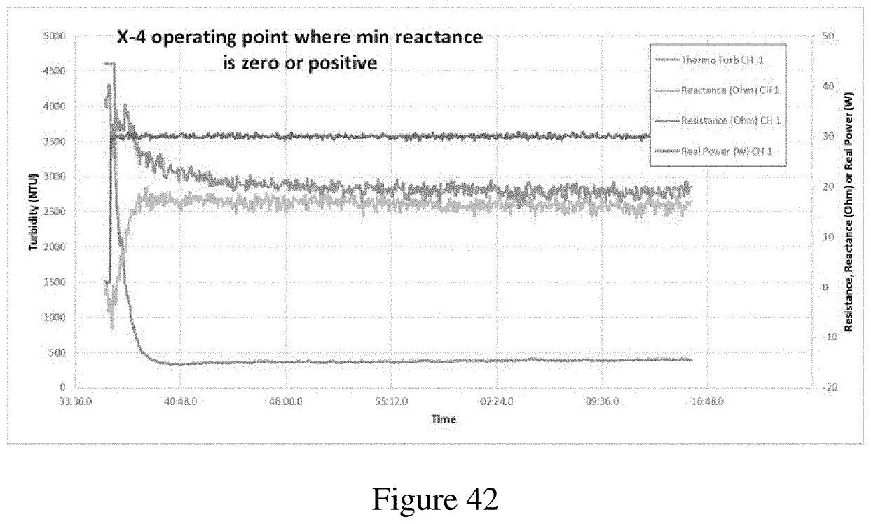

The turbidity performance in FIG. 40 is a significant increase over that illustrated in FIG. 39, with minimum turbidity being often less than 500. The acoustic transducer in this case is operated at a reactance minimum, illustrated in the graph of FIG. 41 at point X-1. Point X-1 represents multimode operation, which can produce axial and lateral forces on particles in the fluid through which the acoustic standing wave passes. These acoustic forces are illustrated in an example in FIG. 36, as well as being shown in FIG. 29. Thus, providing a control technique for operating the acoustic transducer at a reactance minimum can attain desired performance. The desired performance can be attained even at zero phase when operating in multimode, as illustrated with point X-4 in FIG. 41. Point X-4 is a reactance minimum with zero phase, which can achieve desired performance due to multimode operation, unlike the zero-phase planar wave operation. The use of X-4 as an operating point with minimum reactance is illustrated in FIG. 42. As can be seen from the figure, the X-4 operating point provides even better results than the X-1 operating point, even though the X-4 operating point is at about the same level of reactance as the zero phase operating point. This result shows the significant advantages in terms of performance for multimode operation at minimum reactances. These performance benefits are not obtained with zero or planar wave mode of operation for the transducer.

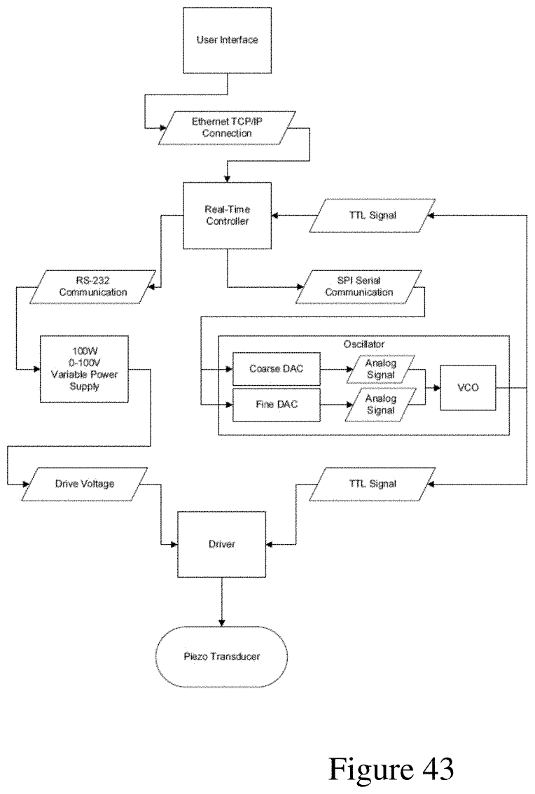

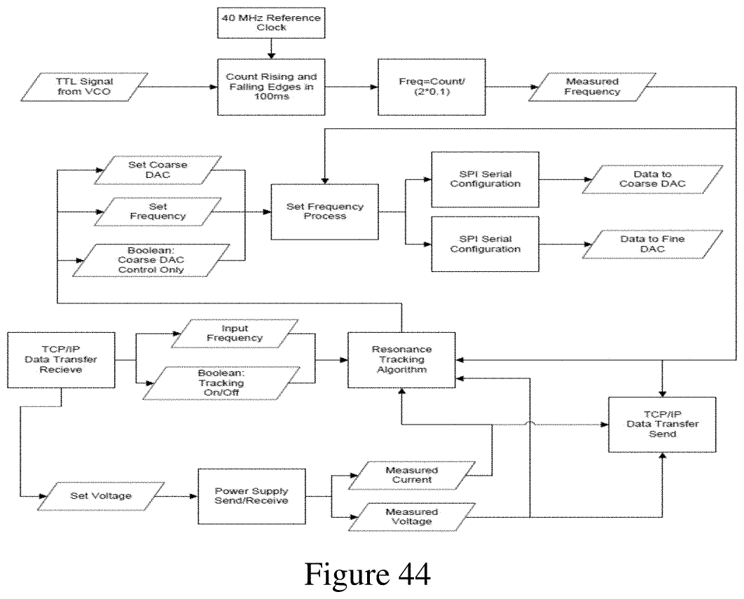

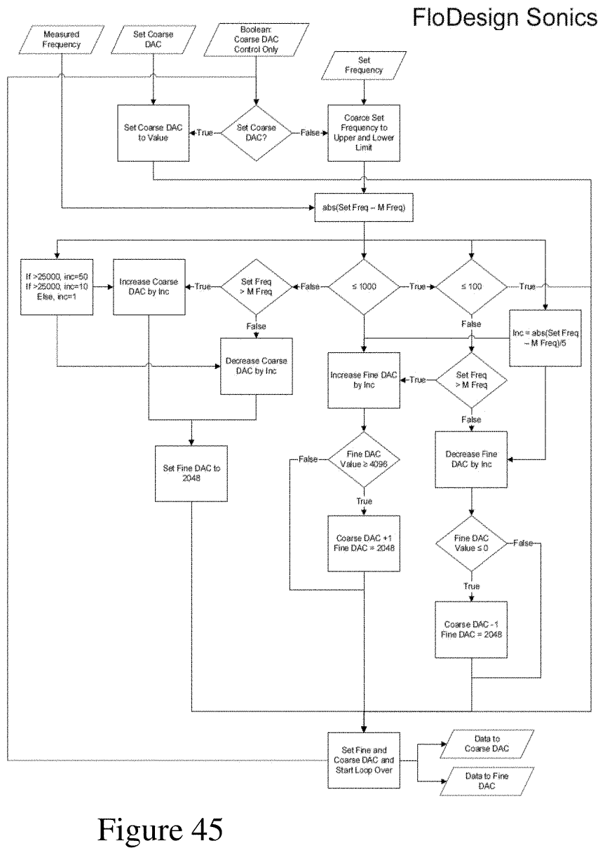

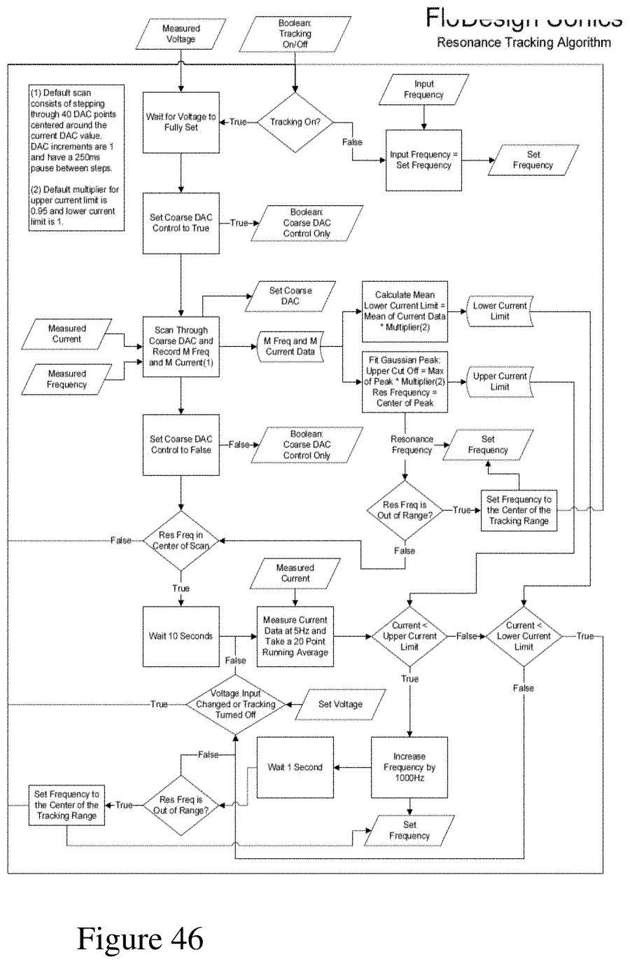

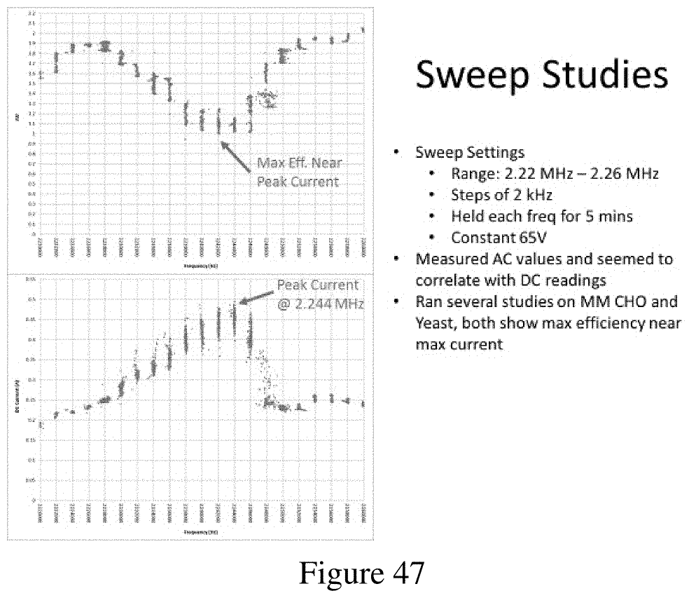

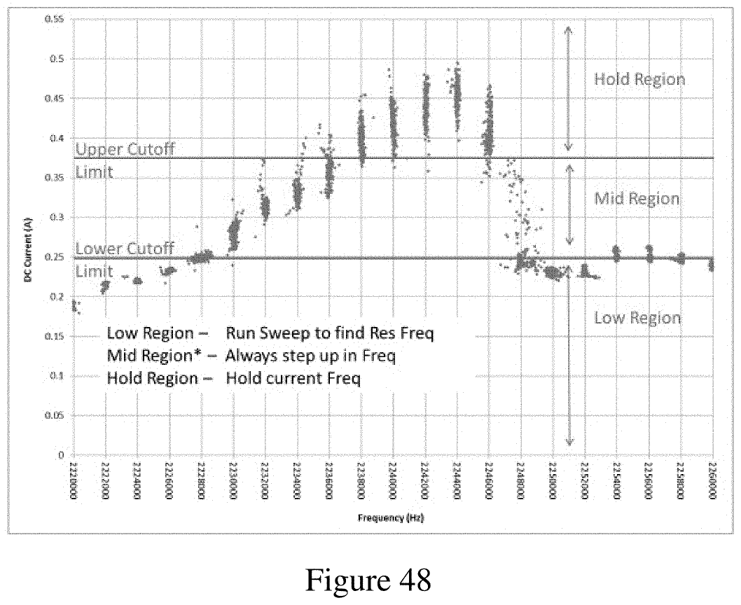

FIGS. 43, 44, 45, and 46 are flowcharts illustrating hardware and software configurations. FIG. 47 shows graphs illustrating a frequency sweep response. FIG. 48 is a graph illustrating regions of operation.

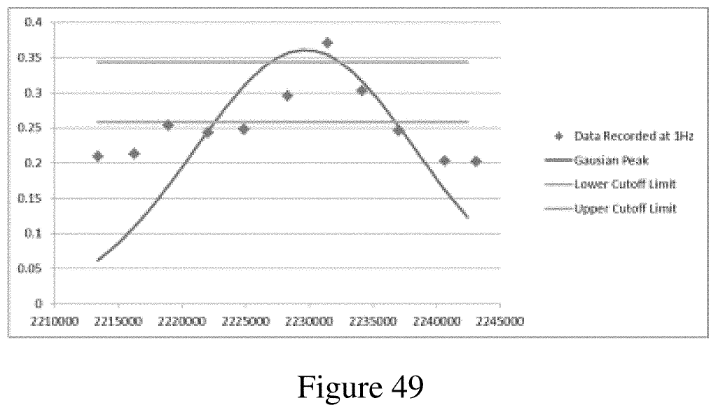

FIG. 49 is a graph illustrating an example control technique. The technique includes a startup and operation process. When the transducer is powered on, the voltage ramps to a desired setpoint. Upon attaining the desired setpoint voltage, a frequency scan is commenced. The frequency scan increments can be set, and may be implemented in a range of from about 1 kHz to about 15 kHz. A starting frequency and ending frequency can be set for the scan. The duration or dwell time for each frequency in the scan range can also be set by an operator, with a nominal duration being 250 ms. With these parameters, the frequency scan may have a total duration of about 10 seconds.

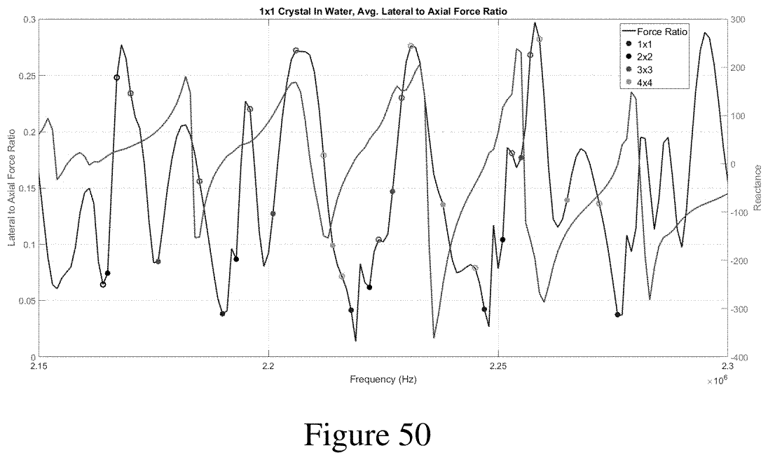

Once the data from the frequency scan is obtained, the measured current information is used to provide a curve that is fitted to a Gaussian curve. The Gaussian curve used to fit the data has a center frequency, or peak, that is the resonance frequency. A value of 95% of the peak current is used to set the upper cut off limit for the current as a safety parameter to avoid damage to the electronic components. An average of the current data for all the frequencies is used to set a lower cut off limit. The graph in FIG. 50 illustrates the curve fitting for the results of the frequency scan.

With the data from the frequency scan, the resonance frequency is determined, and a procedure for monitoring and tracking the desired operating frequency is implemented. The procedure may include wait times for monitoring feedback from the transducer. The wait times can be in a range of from about 0.1 seconds to about 10 seconds. The feedback from the transducer can be sampled or monitored at a range of sample rates, for example from about 1 Hz to about 100 Hz. The number of samples collected during the monitoring or sampling. Can range from about five to several thousand. The sampled data can be used to calculate a running average, which can include points from about five to several thousand for the calculation. The procedure checks the value of the running average, and if it stays above the upper cut off limit, the current operating frequency is used and the system continues to monitor the feedback from the transducer. If the running average drops below the upper cut off limit, the frequency is modified by increasing or decreasing the operating frequency by a certain amount. The amount of increase or decrease can be in the range of from about 0.5 kHz to about 50 kHz, with a nominal value being about 1 kHz. With the frequency change, the feedback from the transducer is monitored to determine if the running average moves in a desired direction, such as above the upper cut off limit. If the running average drops below the lower cut off limit, a new frequency scan is commenced. Upon a change in the voltage of the system, a new frequency scan can be initiated. The frequency tracking range can be set by the user, and may be in a range of from about 2.2 MHz to about 2.26 MHz. A limit on the frequency scan/tracking algorithm is provided if the frequency moves out of the above range, to reset the operating frequency to 2.23 MHz

according to another control implementation, a controller, which may be implemented as an FPGA, acquires samples of feedback from the transducer with two 14-bit analog to digital converters (ADCs) running at 100 MS/s. The FPGA can be configured to processes the samples within 10 ns and/or spread out the calculations over multiple 10 ns cycles using RTL methods.

The ADCs are pipelined devices meaning they can produce one sample per clock cycle using an internal pipeline to que up samples. They can produce samples at 100 MHz and permit the retention of desired signal information that is used for the control process and tracking parameters. The ADCs may have the following specifications: frequency of 100 MS/s (10 ns period), 14-bits, 2Vpp; input resistance of 50 ohms; capable of sampling RF voltage/current.

The sampled data fed back from the transducer can be used to calculate a number of parameters used for the control procedure. Some of the calculations undertaken by the controller based on the feedback data includes apparent power, real power, reactive power, impedance, resistance, reactance, phase angle between voltage and current, real power factor, reactive power factor and RMS voltage and current.

The parameters for acquiring feedback from the transducer may include offsets, scaling and delays to help condition the signals for improved accuracy and ease of calculation. The feedback measured can include the raw voltage and raw current obtained at the sample point. The raw current and voltage samples can be conditioned to be used by the controller in the calculation of desired parameters for the control procedure. The conditioned RF voltage and current obtained from the raw sampling input are input into a calculation module.

The process for phase calculation can include a quadrature operation, and may include the following steps.
a. I-Q Demodulation for both V & I to calculate channel phase.
b. Subtract Iphase−Vphase
c. Unwrap phase to (−180 to 180)
d. Convert output to degrees

The phase calculations can use the following equations.

$${V}_{p h a s e} = {tan}^{- 1} ( \frac{\frac{1}{n} {\sum }_{i = 1}^{n} {( V * sin x )}_{i}}{\frac{1}{n} {\sum }_{i = 1}^{n} {( V * cos x )}_{i}} )$$

$${I}_{p h a s e} = {tan}^{- 1} ( \frac{\frac{1}{n} {\sum }_{i = 1}^{n} {( I * sin x )}_{i}}{\frac{1}{n} {\sum }_{i = 1}^{n} {( I * cos x )}_{i}} )$$

$$Phase Angle = {I}_{p h a s e} - {V}_{p h a s e}$$

The RMS voltage and current can be calculated from the conditioned voltage and current inputs. The voltage and current DC offset can also be calculated based on the feedback data. The following calculations can be performed.

$${V}_{R M S} = \sqrt{( \frac{1}{n} {\sum }_{i = 1}^{n} {( {V}^{2} )}_{i} )}$$

$${I}_{R M S} = \sqrt{( \frac{1}{n} {\sum }_{i = 1}^{n} {( {I}^{2} )}_{i} )}$$

$${V}_{D C} = \frac{1}{n} {\sum }_{i = 1}^{n} {( V )}_{i}$$

$${I}_{D C} = \frac{1}{n} {\sum }_{i = 1}^{n} {( I )}_{i}$$

Power calculations can also be performed using the RMS values and phase angle. Following calculations can obtain the noted power values.

S_{Apparent Power}=V_{RMS}*I_{RMS}

Q Reactive Power=V_{RMS}*I_{RMS}*sin(Phase Angle)

P_{Real Power}=V_{RMS}*I_{RMS}*cos(Phase Angle)

Impedance calculations can also be performed using the RMS values and phase angle. The following calculations can obtain the noted impedance values.

$${Z}_{Impedance} = \frac{{V}_{R M S}}{{I}_{R M S}} \\ {X}_{R e a c t a n c e} = \frac{{V}_{R M S}}{{I}_{R M S}} * sin ( Phase Angle )$$

$${R}_{Resistance} = \frac{{V}_{R M S}}{{I}_{R M S}} * cos ( Phase Angle )$$

The control system may use a proportional-integral (PI) control in a closed loop control setting to provide the control for driving the transducer. A separate PI loop for gain and for frequency can be implemented based on the feedback parameters determined above.

The control system can take advantage of feedback obtained from the transducer based on a frequency sweep function. With this sweep function, the transducer is driven to operate over a range of frequencies, and the feedback for each of the frequencies in the range is collected. The sweep function thus provides a technique for perturbing the system to obtain feedback at different operating points. The control process can use the collected feedback from the sweep function to determine desired operating points and seek to optimize the operation of the system.

The frequency sweep function can accept input to permit a desired frequency sweep to be carried out. The sweep function can, for example, be set to have a number of steps, a given range of frequencies, a step size and/or a given rates of frequency steps. A sample trigger can be used as the trigger to adjust to the next frequency step in the range. The rate at which the frequency is swept, or scanned, can be controlled according to a number of parameters, including the frequency range, the number of steps, multiple samples at a given step or set of steps, to name a few examples.

The control procedure can also implement various protections for the electronic components, including voltage, current and/or power limits. For example, the controller can implement a foldback on the gain amplitude when RMS current or apparent power (VA) reach a defined limit. The fullback can be implemented with two PI controllers for either or both the current and apparent power, with the current and apparent power as the setpoint for their respective PI controllers. The PI controllers can be implemented such that the output of the PI controller is a 0%-100% value that is multiplied by the calculated output gain amplitude value to produce a protected command output gain amplitude value. In one example, the PI controller with the smallest percentage output value is used to determine the protected command output gain amplitude value.

The controller can also provide overcurrent/overpower limitations for the driver, including an RF or inverter section as well as a buck or power converter section. Can be implemented by monitoring RMS current and apparent power. If the monitored parameters exceed setpoint limitations, the driver can be shut down, or the output can be folded back as described above. Various fullbacks can be implemented, including voltage, current, frequency, power, phase, or any other kind of electrical signal related parameter to control for out of range operations.

The above described controller can seek and determine a desired operating setpoint for the transducer and resonant cavity, including operating at desired modes. The desired setpoints can represent optimal operating conditions for capturing particles and/or fluids in the resident cavity, which can be implemented as an acoustic or flow chamber.

FIGS. 50, 51, 52 and 53 are graphs providing plots of various parameters versus frequency. FIG. 50 is a graph with a left-hand scale measuring a ratio of lateral-to-axial forces for various frequencies (blue line), and a right-hand scale measuring reactance (red line). Identified on the ratio graph lines are locations and ranges for various modes of multimode operation. A range of a given mode for multimode operation is identified as existing between open circles, with a primary or dominant frequency for that mode being identified as a solid circle.

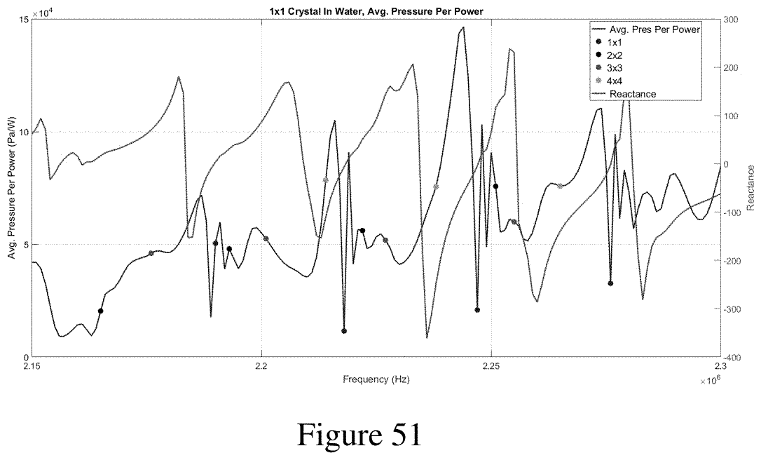

FIG. 51 is a graph with a left-hand scale measuring average pressure per power for various frequencies (blue line), and a right-hand scale measuring reactance (red line). Identified on the pressure graph line are locations and ranges for various modes of multimode operation. A given mode for multimode operation is identified as a circle that a primary or dominant frequency for that mode.

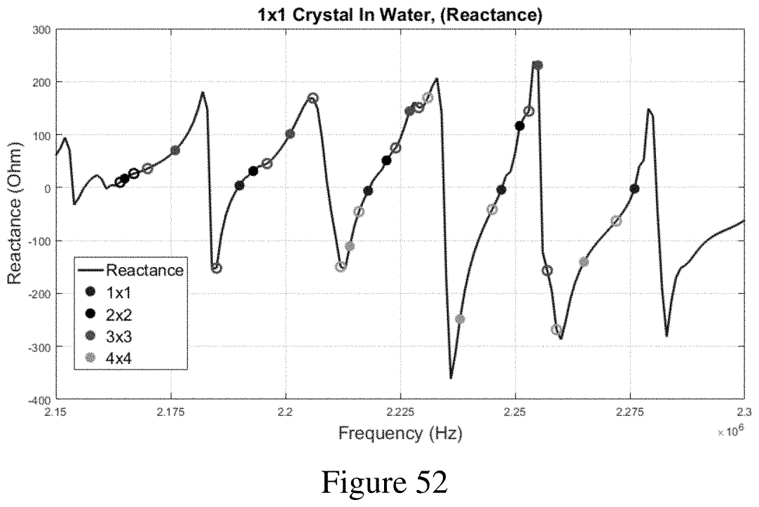

FIG. 52 is a graph showing reactance versus frequency, with a number of modes for multimode operation being identified as locations and ranges on the graph line. A range of a given mode for multimode operation is identified as existing between open circles, with a primary or dominant frequency for that mode being identified as a solid circle.

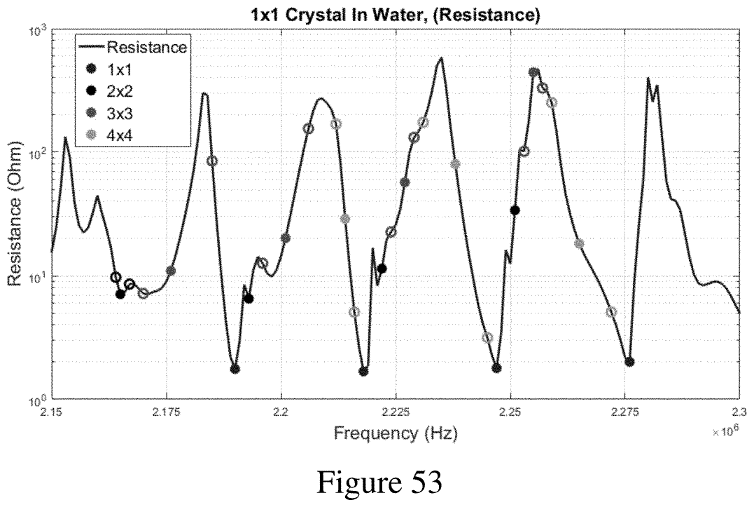

FIG. 53 is a graph showing resistance versus frequency, with a number of modes for multimode operation being identified as locations and ranges on the graph line. A range of a given mode for multimode operation is identified as existing between open circles, with a primary or dominant frequency for that mode being identified as a solid circle.

As can be seen with FIGS. 50-53, multimode operation is strong near minimum reactance. FIG. 50 shows a force ratio plot with a ratio of >0.1 at minimum reactance points. Along with these simulation results, experimental data showing minimum reactance gives the best performance. Note that the tests illustrated in FIGS. 52-55 reflect steady state tests.

The acoustophoretic devices, including that illustrated in FIG. 1 of the present disclosure, can be used in a filter “train,” in which multiple different filtration steps are used to clarify or purify an initial fluid/particle mixture to obtain the desired product and manage different materials from each filtration step. Each filtration step can be optimized to remove a particular material, improving the overall efficiency of the clarification process. An individual acoustophoretic device can operate as one or multiple filtration steps. For example, each individual ultrasonic transducer within a particular acoustophoretic device can be operated to trap materials within a given particle range. In particular, the acoustophoretic device can be used to remove large quantities of material, reducing the burden on subsequent downstream filtration steps/stages. Additional filtration steps/stages can be placed upstream or downstream of the acoustophoretic device. Multiple acoustophoretic devices can be used as well. Desirable biomolecules or cells can be recovered/separated after such filtration/purification.

The outlets of the acoustophoretic devices of the present disclosure (e.g. clarified fluid and concentrated cells), including that illustrated in FIG. 1, can be fluidly connected to any other filtration step or filtration stage. Such filtration steps can include various methods such as depth filtration, sterile filtration, size exclusion filtration, or tangential filtration. Depth filtration uses physical porous filtration mediums that can retain material through the entire depth of the filter. In sterile filtration, membrane filters with extremely small pore sizes are used to remove microorganisms and viruses, generally without heat or irradiation or exposure to chemicals. Size exclusion filtration separates materials by size and/or molecular weight using physical filters with pores of given size. In tangential filtration, the majority of fluid flow is across the surface of the filter, rather than into the filter.

Chromatography can also be used, including cationic chromatography columns, anionic chromatography columns, affinity chromatography columns, mixed bed chromatography columns. Other hydrophilic/hydrophobic processes can also be used for filtration purposes.

Desirably, flow rates through the devices of the present disclosure can be a minimum of 4.65 mL/min per cm² of cross-sectional area of the acoustic chamber. Even more desirably, the flow rate can be as high as 25 mL/min/cm², and can range as high as 40 mL/min/cm² to 270 mL/min/cm², or even higher. This is true for batch reactors, fed-batch bioreactors and perfusion bioreactors, with which the acoustophoretic devices and transducers discuss herein may be used. For example, the acoustophoretic devices may be interposed between a bioreactor and a downstream filtration device, such as those discussed above. The acoustophoretic devices may be configured to be downstream of a filtration device coupled to a bioreactor, and may be upstream of other filtration devices. In addition, the acoustophoretic devices and/or other filtration devices can be configured to have a feedback to the bioreactor.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

1. An acoustophoresis system, comprising:
a chamber for receiving a fluid mixture that includes cells or particles in a fluid;
an ultrasonic transducer coupled to the chamber and configured to be excited to generate an acoustic wave in the chamber,
a driver electrically connected to the ultrasonic transducer and configured to provide an excitation to the ultrasonic transducer to generate the acoustic wave in the chamber; and
a controller electrically connected to the driver and the ultrasonic transducer and configured to receive feedback signals from the ultrasonic transducer and to control the driver.

2. The system of claim 1, wherein the ultrasonic transducer comprises a plurality of transducers, each of the plurality of transducers being electrically connected to a distinct driver.

3. The system of claim 1, wherein the driver further comprises a DC converter and an RF inverter.

4. The system of claim 1, further comprising a capacitor electrically connected between the driver and the ultrasonic transducer.

5. The system of claim 1, further comprising a power resistor electrically connected between the driver and the ultrasonic transducer.

6. The system of claim 1, further comprising the controller being configured to determine frequencies where anti-resonance, and reactance minima and maxima occur based on the feedback signals.

7. The system of claim 1, further comprising the controller being configured to select a frequency associated with a reactance minimum or maximum based on the feedback signals.

8. A method for controlling an acoustic transducer, comprising:
determining an anti-resonance frequency of the acoustic transducer;
determining a reactance minimum or maximum adjacent to the anti-resonance frequency; and
providing a power signal to the acoustic transducer with the frequency substantially of the reactance minimum or maximum.

9. The method according to claim 8, further comprising:
scanning a frequency range for a new reactance minimum or maximum; and
adjusting the frequency of the power signal to a new frequency associated with the new reactance minimum or maximum.

10. The method of claim 8, further comprising:
receiving feedback signals from the acoustic transducer; and
determining an electrical power consumed by the acoustic transducer based on the feedback signals.

11. The method of claim 10, further comprising controlling one or more of a voltage, a current or a frequency of the power signal provided to the acoustic transducer to control electrical power consumed by the acoustic transducer.

12. The method of claim 8, further comprising:
receiving feedback signals from the acoustic transducer; and
decomposing the feedback signals into in-phase and quadrature-phase components.

13. The method of claim 12, further comprising determining phase angle and reactance based on the in-phase and quadrature-phase components.

14. The method of claim 8, further comprising sampling voltage and current of the acoustic transducer with pipelined analog-to-digital converters.

15. The method of claim 8, further comprising initiating a frequency scan based on one or more of a timed interval or an event.

16. The method of claim 15, further comprising receiving parameters of the frequency scan that include one or more of a frequency range, a frequency step size or a frequency step time interval.

17. The method of claim 8, further comprising determining the phase angle of the impedance of the ultrasonic transducer.

18. A device for controlling an acoustic transducer, comprising:
a modular controller for implementing a control scheme;
a power section connected to the controller for supplying power to the acoustic transducer, and
a feedback section interposed between the acoustic transducer and the controller to provide feedback to the controller for the operating parameters of the acoustic transducer.

19. The device of claim 18, wherein the power section further comprises a power converter and an RF inverter.

20. The device of claim 18, wherein the controller further comprises a processing engine configured to receive feedback signals, determine a minimum or maximum reactance from the feedback signals, and provide control signals to the power section to control one or more of voltage, current, or frequency of an output of the power section.
